(19) United States
(12) Patent Application Publication
(10) Pub. No.: US 2003/0003048 A1
(43) Pub. Date: Jan. 2, 2003

(54) Diagnostic imaging compositions, their methods of synthesis and use

(76) Inventors: Chun Li, Missouri City, TX (US); Xiaoxia Wen, Houston, TX (US); Qing-Ping Wu, Pearland, TX (US); Sidney Wallace, Bellaire, TX (US); Lee M. Ellis, Houston, TX (US)

Correspondence Address: Lori D. Stiffler, Baker Botts L.L.P., One Shell Plaza, 910 Louisiana Street, Houston, TX, 77002-4995, US

(21) Appl. No.: 10/126,216

(22) Filed: Apr. 19, 2002

Related U.S. Application Data
(60) Provisional application No. 60/286,453, filed on Apr. 26, 2001.
(60) Provisional application No. 60/334,969, filed on Dec. 4, 2001.
(60) Provisional application No. 60/343,147, filed on Dec. 20, 2001.

(51) Int. Cl. A61K 51/00; C07K 16/46; C07H 1/00; C07K 14/00

(52) U.S. Cl. 424/1.49; 530/391.1; 536/123; 530/350

(57) ABSTRACT

Conjugate molecules comprising a ligand bonded to a polymer are disclosed. One such conjugate molecule comprises a ligand bonded to a polymer, a chelating agent bonded to the polymer, and a radioisotope chelated to the chelating agent. The conjugate molecules may be useful in detecting and/or treating tumors or biological receptors. These conjugate molecules may be synthesized without the necessity of preactivation of the ligand using an SCN-polymer-chelating agent precursor. Conjugate molecules incorporating an annexin V ligand are particularly useful for visualizing apoptotic cells. Conjugate molecules incorporating a C225 ligand are particularly useful for targeting tumors expressing EGFR.

### CROSS REFERENCE TO RELATED APPLICATIONS

This application claims the benefit of: U.S. Provisional Patent Application No. 60/286,453, entitled “Methods for Visualizing Tumors Using a Radioisotope Conjugate” filed Apr. 26, 2001; U.S. Provisional Patent Application No. 60/334,969, entitled “Therapeutic Agent/Ligand Conjugate Compositions and Methods of Use” filed Dec. 4, 2001; and U.S. Provisional Patent Application No. 60/343,147, entitled “Diagnostic Imaging Compositions, Their Methods of Synthesis and Use” filed Dec. 20, 2001, all three of which are hereby incorporated herein by reference in their entirety. This application is related to U.S. Patent Application Ser. No. ______, entitled “Therapeutic Agent/Ligand Conjugate Compositions, Their Methods of Synthesis and Use,” filed Apr. 19, 2002, inventors Chun Li, et al., which is hereby incorporated herein by reference in its entirety.

### RIGHTS IN THE INVENTION

This invention was made, in part, with United States Government support under NIH Cancer Center Support Grant NIH 90810, and the United States Government may therefore have certain rights in the invention.

### BACKGROUND OF THE INVENTION

1. Field of the Invention

This invention relates to compositions useful in the diagnosis and treatment of cancer and other diseases, and, more specifically, to compositions comprising diagnostic agents (e.g., radioisotopes) and other compounds conjugated to ligands, useful for detecting, treating, or monitoring treatment of tumors and other tissues with biological receptors. The invention also relates to methods for synthesizing and using such compositions.

2. Description of the Background

Visualizing tumors and their response to therapy are important steps in the diagnosis and therapy of cancers in humans and in other animals. Various methods have been employed in the art. These methods include nuclear imaging, such as radioimmunoscintigraphy and receptor-mediated imaging.

Most radiolabeled monoclonal antibodies used for radioimmunoscintigraphy and proteins for receptor-mediated imaging suffer from two key limitations: significant liver uptake, and rapid clearance from the body after administration. These properties can lead to obscured images due to high background activity, especially when imaging diseases in the abdomen, or to weak target activity because the ligands do not have sufficient time to interact with antigens or receptors.

Thus, there is a need for new and improved compositions and methods for the visualization and treatment of tumors and other diseases, to compositions and methods for monitoring the response of tumors to therapy, and to methods for synthesizing such compositions.

### SUMMARY OF THE INVENTION

The present invention overcomes problems and disadvantages associated with current therapeutic and diagnostic agents, and provides novel compositions for the diagnosis, treatment, and evaluation of treatment of tumors and other diseases. Some preferred compositions selectively bind to tumor cells expressing certain receptors, such as EGFR. Some preferred compositions allow for the non-invasive visualization of apoptotic cells in tumors and other tissues and thus, their response to therapy. The invention also provides novel methods for synthesizing these compositions. The new compositions preferably have longer in vivo half lives, reduced liver uptake, and higher imaging ratios.

Accordingly, one embodiment is directed to a conjugate molecule comprising: a ligand bonded to a polymer; a chelating agent bonded to the polymer; and radioisotope chelated to the chelating agent. Preferably, the ligand is covalently bonded to the polymer and the chelating agent is covalently bonded to the polymer.

Still another embodiment is directed to a composition comprising any of the conjugate molecules described herein and a pharmaceutically acceptable carrier.

Another embodiment of the invention is directed to a method for synthesizing a ligand-polymer-chelating agent-diagnostic agent conjugate molecule comprising: providing an SCN-polymer-chelating agent precursor, wherein the polymer is covalently bonded to the chelating agent and the SCN group is covalently bonded to the polymer; combining a ligand with the SCN-polymer-chelating agent precursor to form a ligand-polymer-chelating agent conjugate; and combining the ligand-polymer-chelating agent conjugate with a diagnostic agent to form the ligand-polymer-chelating agent-diagnostic agent conjugate molecule. Preferably, in the resulting conjugate molecule, the ligand is covalently bonded to the polymer, the chelating agent is covalently bonded to the polymer, and the diagnostic agent is chelated to the chelating agent. Preferably, the ligand comprises a primary amino group.

Another embodiment is directed to a method for synthesizing a conjugate molecule comprising: providing a polymer conjugate-SCN precursor, wherein the SCN group is covalently bonded to the polymer conjugate; and combining a ligand with the polymer conjugate-SCN precursor to form a ligand-polymer conjugate molecule in which the ligand is covalently bonded to the polymer. The polymer conjugate may comprise a polymer covalently bonded to a chelating agent. The method may further comprise the it step of combining the ligand-polymer conjugate molecule with a diagnostic agent to form a ligand-polymer-chelating agent-diagnostic agent conjugate molecule. Preferably, the ligand comprises a primary amino group.

The invention also includes various methods for synthesizing conjugate molecules of the invention involving preactivation or other preparation of the ligand or polymer. One such method for synthesizing a conjugate molecule comprises the steps of: providing a polymer conjugate, wherein the polymer conjugate comprises at least one thio (SH) group covalently conjugated to the polymer conjugate; providing a ligand, the ligand comprising at least one thio reactive group; and combining the polymer conjugate and the ligand to form a ligand-polymer conjugate molecule, in which the ligand is preferably covalently bonded to the polymer by a thioether (S—C) bond.

The methods of the invention are not limited to processes where the ligand includes the thio reactive group. For example, the invention also includes methods for synthesizing a conjugate molecule in which either the ligand or the polymer conjugate has the thio reactive group. One such method comprises the steps of: providing a polymer conjugate and a ligand, wherein one of the polymer conjugate or the ligand comprises a thio group, and the other of the polymer conjugate or the ligand comprises a thio reactive group; and combining the polymer conjugate and the ligand to form a ligand-polymer conjugate molecule. The ligand is preferably covalently bonded to the polymer by a thioether (S—C) bond.

The invention also includes therapeutic applications for the compositions of the invention. For example, one embodiment is directed to a method of treating a patient suspected of having a tumor comprising administering a therapeutically effective amount of a conjugate molecule to the patient. The conjugate molecule comprises a ligand bonded to a polymer, a chelating agent bonded to the polymer, and a radioisotope chelated to the chelating agent. The ligand has affinity for and selectively binds to the tumor.

Another embodiment is directed to a method for selectively delivering a diagnostic agent to apoptotic cells in a patient comprising: administering a conjugate molecule to the patient having apoptotic cells. The conjugate molecule comprises a ligand bonded to a polymer, a chelating agent bonded to the polymer, and a radioisotope chelated to the chelating agent. The ligand is annexin V.

Another embodiment is directed to a method of visualizing tumors. This method comprises the steps of administering a conjugate molecule to a patient suspected of containing a tumor, and detecting the conjugate molecule. The conjugate molecule comprises a ligand bonded to a polymer, a chelating agent bonded to the polymer, and a radioisotope chelated to the chelating agent. The ligand has affinity for and selectively binds to the tumor.

Still another embodiment is directed to a method for imaging or visualizing apoptotic cells in a patient comprising the steps of: administering a conjugate molecule to the patient having apoptotic cells, wherein the conjugate molecule comprises a ligand bonded to a polymer, wherein the ligand is annexin V, a chelating agent bonded to the polymer, and a radioisotope chelated to the chelating agent; and detecting the conjugate molecule.

Another method for visualizing tumors or apoptotic cells comprises the steps of administering a conjugate molecule to a patient suspected of having a tumor or apoptotic cells; and detecting the conjugate molecule. In this embodiment, the conjugate molecule comprises a ligand bonded to a polymer and a near-infrared dye bonded to the polymer. Preferably, near-infrared dye is ICG (indocyanine green) or an ICG derivative. The ligand has affinity for and selectively binds to the tumor or apoptotic cells. The detecting step may comprise detection of the near-infrared dye by a near-infrared camera.

Other objects and advantages of the invention are set forth in part in the description which follows, and, in part, will be obvious from this description, or may be learned from the practice of the invention.

### DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Synthetic scheme for the synthesis of PEG-modified antibodies according to one embodiment of the invention.

FIG. 2. Graph showing receptor specificity of ¹¹¹In-DTPA-PEG-C225 and ¹¹¹In-DTPA-C225.

FIG. 3. Plot of blood radioactivity against time.

FIG. 4. Whole body scintigram of mouse treated with ¹¹¹In-DTPA-C225.

FIG. 5. Whole body scintigram of mouse treated with ¹¹¹In-DTPA-PEG-C225.

FIG. 6. Whole body scintigram of mouse treated with 1:30 ¹¹¹In-DTPA-PEG-C225.

FIG. 7. Graph showing radioactivity in tumors (tumor to whole body ratio per pixel).

FIG. 8. Graph showing radioactivity in tumors (tumor to liver ratio per pixel).

FIG. 9. Synthetic scheme for the synthesis of DTPA-PEG-annexin V according to one embodiment of the invention.

FIG. 10. Purification of DTPA-PEG-annexin V by ion exchange chromatography.

FIG. 11. SDS-PAGE of isolated products from the reaction between annexin V and SCN-PEG-DTPA at corresponding molar ratios of 1:60 and 1:30, respectively.

FIG. 12. Radio-gel permeation chromatography of (A) 1:60 prep ¹¹¹In-DTPA-PEG-annexin V, (B) 1:30 prep ¹¹¹In-DTPA-PEG-annexin V, and (C). ¹¹¹In-DTPA-PEG.

FIG. 13. Bar graph showing apoptotic index after treatment with 1.0 uM Ara-C as quantified by flow cytometry analysis using annexin V-FITC as fluorescent probe.

FIG. 14. Bar graph showing binding of ¹¹¹In-DTPA-PEG-annexin V to Ara-C treated cells.

FIG. 15. Blood activity-time curve (A) and biodistribution (B) of ¹¹¹In-DTPA-PEG-annexin V and ¹¹¹In-DTPA-annexin V.

FIG. 16. Bar graph showing tissue distribution of ¹¹¹In-DTPA-PEG-annexin V in untreated control mice.

FIG. 17. Bar graph showing distribution of ¹¹¹In-DTPA-PEG-annexin V in mice treated with PG-TXL on day 4.

FIG. 18. Bar graph showing distribution of ¹¹¹In-DTPA-PEG-annexin V in mice treated with C225 on day 4.

FIG. 19. Bar graph showing percentage of apoptotic cells determined histologically.

FIG. 20. Graph showing correlation between apoptotic index measured by histological examination and tumor uptake of radiolabeled annexin V.

FIG. 21. Graph showing correlation between radioactivity in autoradiographs and fluorescent intensity in TUNEL stained slides.

### DESCRIPTION OF THE INVENTION

The present invention is directed to novel conjugates useful for the visualization and targeted therapy of tumors and other target tissues, including conjugates useful for monitoring the response of tumors and other tissues to therapy. The invention is also directed to novel methods of synthesizing and using such conjugates.

As noted, most radiolabeled monoclonal antibodies (mAb) for radioimmunoscintigraphy and proteins for receptor-mediated imaging suffer two major limitations: (1) significant liver uptake; and (2) rapid clearance from the body after administration. These properties either lead to obscured images due to strong background activity, particularly for diseases in the abdomen, or to weak target activity because the ligands do not have enough time to interact with antigens or receptors.

It has been discovered that the imaging properties of ligands (e.g., monoclonal antibodies (mAb) or proteins) attached to metal chelators can be enhanced by using a polymer linker between the ligand and chelator. By introducing a polymer linker (such a polyethylene glycol (PEG) linker) between the metal chelator and the ligand, the resulting construct, after being labeled with a radioisotope, may be used in radioscintigraphy to obtain optimized nuclear images.

PEG is an uncharged, hydrophilic, non-toxic, linear polymer. These characteristics make it particularly useful for protein/ligand modification. Modification of a ligand with PEG according to preferred embodiments of the invention may confer a number of advantages, such as improved biocompatibility, reduced liver uptake, increased circulation half-life, decreased immunogenicity, increased resistance to proteolysis and enhanced solubility and stability. It is believed that the reduction in immunogenicity is due to the steric hindrance by the PEG strands preventing recognition of foreign protein by the immune system. The PEG-modification is believed to interfere with the recognition of foreign particles and proteins by the reticuloendothelial system, and thus reduces the liver uptake of the particles and proteins. Further, attachment of metal chelators such as DTPA through a PEG linker instead of directly attaching DTPA and PEG sequentially to mAb is expected to reduce chemical manipulation of mAb, and maximize retention of the mAb's receptor-binding affinity. Improved tumor-to-normal tissue ratio allows for optimized tumor imaging. Further, using the described methods, the modified mAb can be synthesized in a reproducible manner, and the degree of substitution easily controlled and the products conveniently characterized.

As shown in the Examples, novel ligand-polymer-chelating agent-radioisotope constructs were synthesized. In one construct, mAb C225 was used as a model protein, diethylenetriaminepentaacetic acid (DTPA) labeled with Indium-111 (¹¹¹In) was used as a model radioisotope chelator, and PEG was used as the polymer linker. In the resulting conjugate, one end of the PEG linker molecule was attached to C225 and another end was attached to ¹¹¹In-DTPA.

C225 is an anti-epidermal growth factor receptor (EGFR or EGF receptor) antibody. Specifically, C225 is a human-mouse chimeric monoclonal antibody directed against human EGFR. EGFR is a transmembrane glycoprotein with an intracellular tyrosine kinase domain. EGFR is overexpressed on the cells of over one-third of all solid tumors, including bladder, breast, colon, ovarian, prostate, renal cell, squamous cell, non-small cell lung, and head and neck carcinomas. C225 specifically binds to the external domain of the receptor with an affinity comparable to the natural ligand. C225 is believed to inhibit both the initiation and propagation of EGFR positive cells, therefore stopping tumor growth. C225 has been demonstrated to inhibit the proliferation of a variety of human cancer cells stimulated by the transforming growth factor-α (TGF-α) and EGFR autocrine loop.

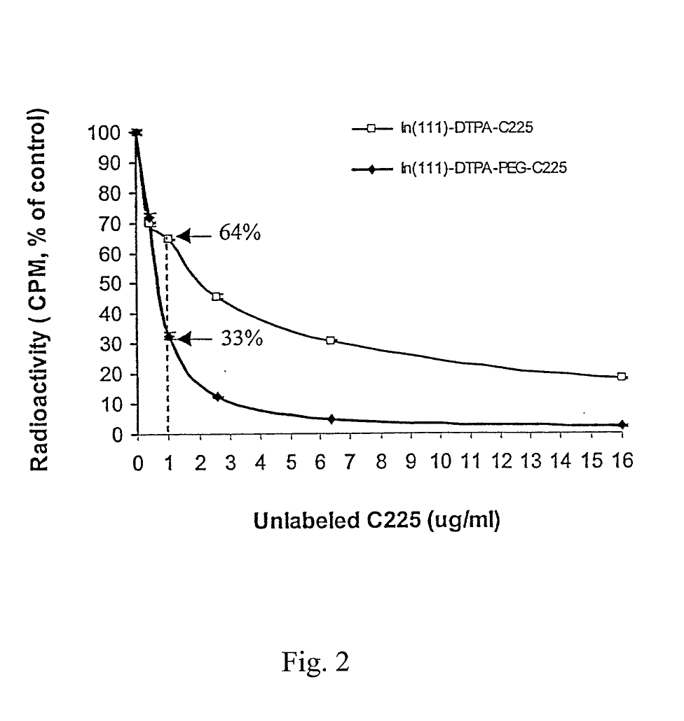

In vitro studies (immunoprecipitation, competitive binding, and cytotoxicity assays) demonstrated that the constructs retained C225's binding affinity. Specifically, MDA-MB-468 human breast adenocarcinoma cells overexpressing EGFR were incubated with 1 μg/ml of 1:30 ¹¹¹In-DTPA-PEG-C225 or ¹¹¹In-DTPA-C225 in the presence of native C225 mnAb. As shown in FIG. 2, the binding of both radiomolecules to the cells was EGFR specific, because they could be displaced by native C225. Both ¹¹¹In-DTPA-PEG-C225 and ¹¹¹In-DTPA-C225 were able to compete with native C225 mAb for the binding to MDA-468 cells. In the presence of 1 μg/ml of native C225 mAb (equal to the amount of the labeled molecules), 64% of ¹¹¹In-DTPA-C225 and 33% of 1:30 ¹¹¹In-DTPA-PEG-C225 were competitively bound to the cells. These data indicate that 1:30 DTPA-PEG-C225 retained about half of its binding affinity as compared to DTPA-C225. ¹¹¹In-DTPA-PEG-C225 was almost fully displaced by a 16-fold excess of C225, whereas only 80% of ¹¹¹In-DTPA-C225 was displaced by a 16-fold excess of C225 and could not be fully displaced even by a 40-fold excess of C225. The remaining 20% of cell-associated ¹¹¹In-DTPA-C225 represents non-specific binding. These results indicate that modification of C225 with PEG according to the invention reduced the non-specific interaction of the antibody.

To further assess the receptor-binding affinity of DTPA-PEG-C225 conjugates, the capacity of DTPA-PEG-C225 conjugates to immunoprecipitate EGFR was investigated using a human vulvar squamous carcinoma cell line A431 that expresses high level of EGFR. The A431 cells were exposed to mAb C225 or each of the three DTPA-PEG-C225 conjugates with different degrees of substitution for half an hour and then were lysated. The antibody-receptor immunocomplexes were collected by protein A-Sepharose beads. Western blotting techniques were applied to assess the levels of EGFR immunoprecipitated by mAb C225 and DTPA-PEG-C225 derivatives. The levels of EGFR revealed by western blotting represent the amounts of C225 bound to the receptor. As such, the EGFR levels revealed the receptor-binding capacity of C225 and its derivatives. In the immunoprecipitation study, all three DTPA-PEG-C225 molecules (1:10, 1:20 and 1:40) with 25%, 40% or 70% amino groups in C225 substituted, maintained specific binding capacity to the EGFR in A431 cells. The levels of EGFR immunoprecipitated by C225 derivatives decreased with the increase of degree of substitution, which indicated that the binding affinity of the derivatives decreased with the increase of PEG-modification.

A DiFi cell line was used to evaluate in vitro antitumor activity of DTPA-PEG-C225 conjugates. Blockage of DiFi cell growth by native C225, free DTPA-PEG and DTPA-PEG-C225 conjugates was evaluated. The viable cell numbers resulting from treatment with DTPA-PEG-C225 conjugates were remarkably reduced. There was no obvious difference of blocking capacities between DTPA-PEG-C225 derivatives with different degrees of modification. The results also showed that the tumor cell inhibition capacities of PEG-modified antibodies were similar to that of intact C225 mAb. DiFi cells were also treated with DTPA-PEG under the same conditions as for C225 and its conjugates. PEG-DTPA had no effect on DiFi cell growth.

In vivo imaging studies of the construct also yielded favorable results. The in vivo pharmacokinetic and gamma imaging properties of ¹¹¹In-DTPA-PEG-C225 were compared to that of ¹¹¹In-DTPA-C225. ¹¹¹In-DTPA-PEG-C225 was shown to be less widely distributed to normal tissues than ¹¹¹In-DTPA-C225, which may be due to reduced non-specific binding of C225 by PEG-modification.

Tumors of A431 and MDA468 xenografts expressing high levels of EGFR were clearly visualized with ¹¹¹In-DTPA-PEG-C225, while tumors of MDA435 xenograft that express low levels of EGFR were barely visible. In A431 tumor, the tumor uptakes of ¹¹¹In-DTPA-PEG-C225 were similar to that of ¹¹¹In-DTPA-C225. The liver uptakes, however, were reduced by almost 50% for ¹¹¹In-DTPA-PEG-C225 as compared to ¹¹¹In-DTPA-C225. Blocking EGFR by preinjection of native C225 reduced uptakes of ¹¹¹In-DTPA-PEG-C225 in both tumor and liver. The tumor-to-blood ratios in mice with A431 and MDA468 tumors dropped 63% and 53%, respectively, when unlabeled C225 was preinjected. In contrast, the tumor-to-blood ratio in mice with MDA435 tumor did not change significantly.

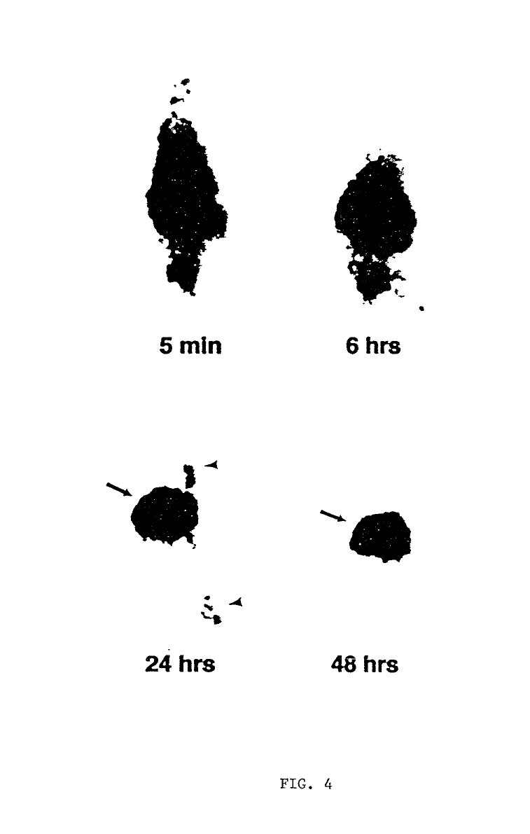

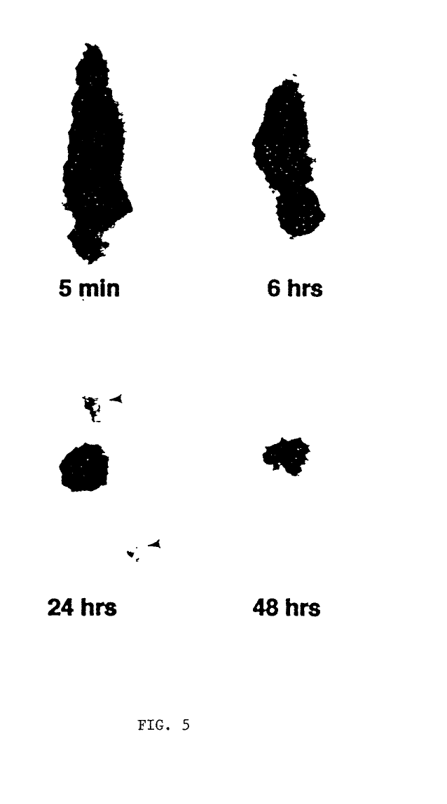

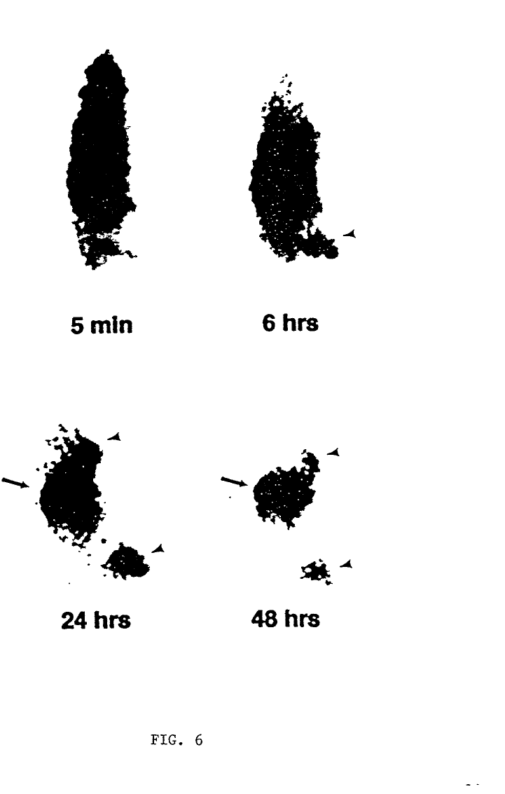

Specifically, nude mice bearing A431 xenografts in chest and right hindlimb were injected with ¹¹¹In-DTPA-C225, 1:10 ¹¹¹In-DTPA-PEG-C225 or 1:30 ¹¹¹In-DTPA-PEG-C225. Whole body gamma scintigrams of mice obtained at different time intervals were obtained. Immediately after injection of each radiotracer, images showed the highest activity in the central location, which is attributable to the cardiac blood pool, the liver and spleen. While activity in the liver of mice injected with ¹¹¹In-DTPA-C225 dominated the images at 24 hours and 48 hours (FIG. 4), significant reduction of radioactivity in the liver was seen with ¹¹¹In-DTPA-PEG-C225 derivatives, particularly at 24 hours and 48 hours (FIGS. 5 and 6). Tumors in right hindlimb and in chest at the site of xenografts were visualized at 24 hours with all three C225 radiotracers. Tumors in mice injected with 1:30 ¹¹¹In-DTPA-PEG-C225 was more clearly visualized than with the other two radiotracers, particularly at 48 hours (FIG. 6).

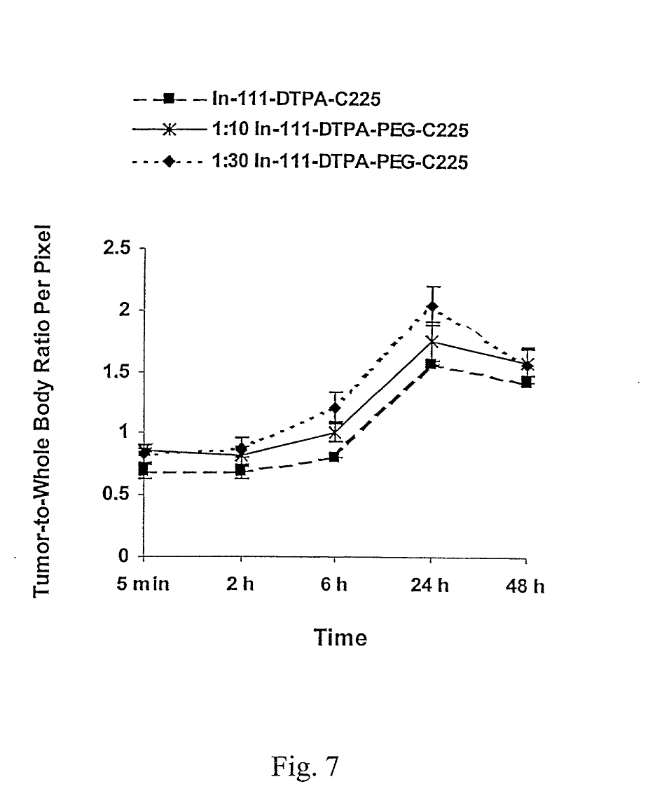

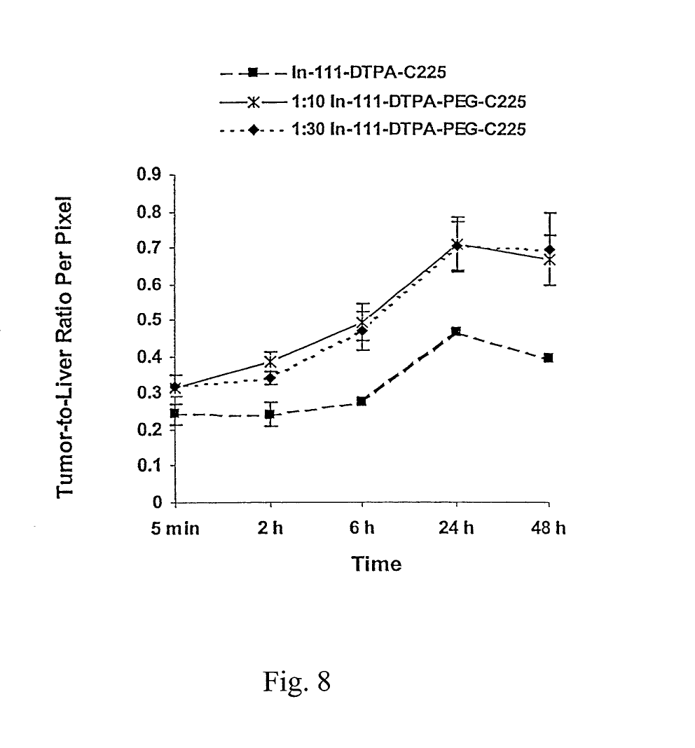

The above observations were confirmed by image quantification. FIG. 7 shows the radioactivity in tumors (both in chest and in hindlimb) expressed as tumor-to-whole body ratio per pixel obtained from sequential gamma camera images at different time intervals. All three radiotracers demonstrated increased tumor radioactivity relative to the whole body background over time; the radioactivity reached the maximum at 24 hours. FIG. 8 presents tumor-to-liver ratio per pixel as a function of time. PEG-modified C225 had significantly higher tumor-to-liver ratios than C225 without PEG at each time point (P<0.05), and the values appeared to increase over time. There was no significant difference in tumor-to-whole body radioactivity ratios with the three radiotracers (p>0.05) (FIG. 7). Therefore, the increase of tumor-to-liver ratios of PEG-modified C225 was due to the decrease of hepatic uptake. Dissection analysis at 48 hours confirmed the findings revealed by the images. Tumor uptake, expressed as percentage of injected dose per gram of tumor, showed no significant difference between 1:30 ¹¹¹In-DTPA-PEG-C225, 1:10. ¹¹¹In-DTPA-PEG-C225 and ¹¹¹In-DTPA-C225. No obvious difference was observed in muscle uptake. The uptake of PEG-modified C225 in the liver, however, was greatly reduced as compared to C225 without PEG (data not shown).

To investigate whether the uptake of DTPA-PEG-C225 in the tumor was mediated through specific interaction of the antibody with the EGFR, each mouse bearing A431 xenografts was preinjected with 1 mg of native C225 to block the EGFR. Twenty hours later, 10 μg of 1:30 ¹¹¹In-DTPA-PEG-C225 was administered intravenously. The γ-camera images showed suppression of tumor uptake of ¹¹¹In-DTPA-PEG-C225 by C225 pretreatment. Dissection analysis performed at 48 hours after injection of 1:30 ¹¹¹In-DTPA-PEG-C225 showed that preinjection of C225 significantly reduced the tumor-to-blood ratio, as well as liver-to-blood ratio, of ¹¹¹In-DTPA-PEG-C225. The results were in agreement with data obtained from γ-images.

To further demonstrate that ¹¹¹In-DTPA-PEG-C225 can specifically localize in tumors that overexpress EGFR, A431, MDA-468 and MDA-435 human tumor xenografts were used in an imaging study. A431 vulvar squamous cell line and MDA468 breast adenocarcinoma cell line express high levels of EGFR, 2×10⁶ receptors per cell and 3×10⁵ receptors per cell, respectively. In contrast, MDA435 breast adenocarcinoma cell line expresses low level of EGFR. Nude mice bearing human tumor xenografts received 10 μg/mouse of 1:30 ¹¹¹In-DTPA-PEG-C225 (50 μCi). Images obtained at 24 hours demonstrated higher accumulation of the radiotracer in tumors at the sites of A431 and MDA468 xenografts than MDA435 xenograft. Tumors were barely seen for MDA435 model. Dissection analysis performed at 48 hours after injection of 1:30 ¹¹¹In-DTPA-PEG-C225 demonstrated that the tumor-to-blood ratios for A431 and MDA468 xenografts were significantly higher than that for MDA435 (P=0.009 for A431 model and P<0.001 for MDA468 model, respectively). Preinjection of C225 20 hours before administration of 1:30 ¹¹¹In-DTPA-PEG-C225 significantly reduced the tumor-to-blood ratios for A431 (P=0.04) and MB468 (P<0.001) tumors, but not for MB435 tumor (P=0.3).

The foregoing findings demonstrate that the ¹¹¹In-DTPA-PEG-C225 construct selectively localizes to tumors that express high numbers of EGFR. PEG-modification of mAb C225 reduced non-specific interaction and significantly reduced the liver uptake, resulting in improved visualization of tumors with EGFR. Thus, by using a polymer linker, such as PEG, between the mAb or proteins and metal chelator, the imaging characteristics of the mAb- or protein-based scintigraphic agents were optimized. By attaching the metal chelator to one end of PEG molecule rather than directly to the protein/mAb molecules, the retention of receptor binding affinity was maximized, leading to improved imaging properties.

Taken together, the data demonstrate that modification of antibodies with PEG can reduce liver uptake of the antibodies and optimize γ-imaging properties. These improvements make PEG-modified proteins according to the invention useful RIS agents for receptor imaging. Coupled with appropriate radionuclides, the constructs of the invention may be used for RIT for the treatment of cancers.

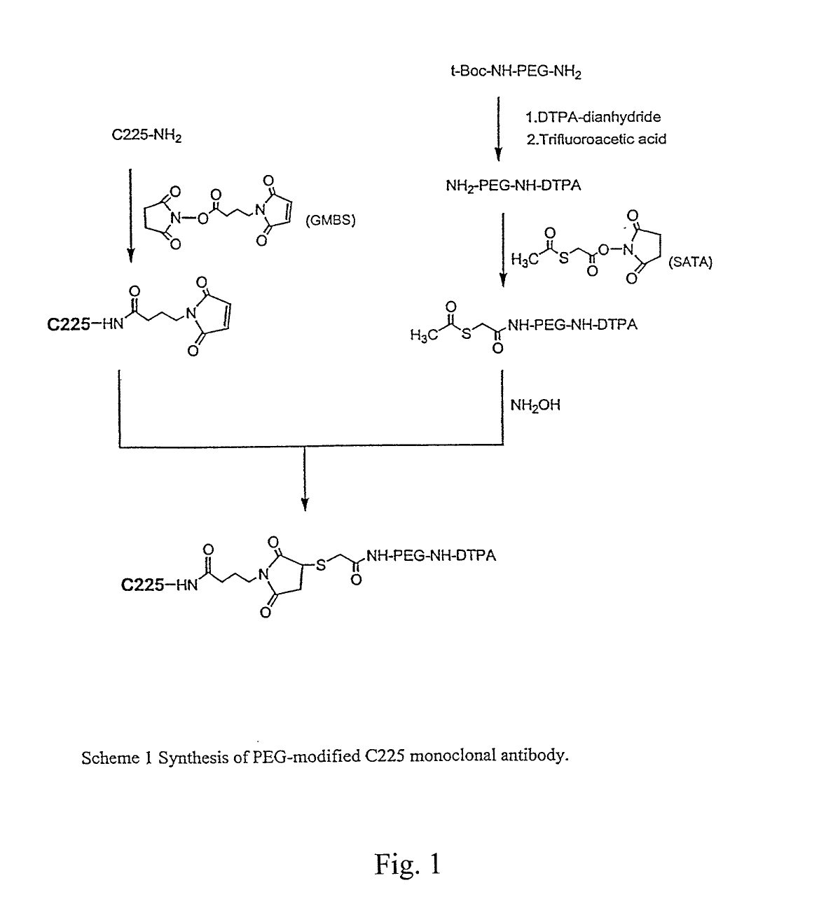

The C225 conjugate molecule used in Examples 1-16 was synthesized according to the scheme set out in FIG. 1. Although the reaction between sulfhydryl and maleimide is highly selective, it requires preactivation of the ligand (see Example 5). This necessitates an additional purification step. Further, the number of PEG molecules attached to the proteins may not necessarily agree with the number of amino groups that have been modified with maleimide, making it difficult to control the degree of PEGylation.

It has further been discovered that conjugate molecules according to the invention may alternately be synthesized using a new, more simplified procedure. By introducing an NH₂ reactive group, e.g., an SCN group, at one end of the PEG molecule, PEGylation of ligand proteins may be achieved by simply mixing SCN-PEG-DTPA with the ligand protein without the necessity of preactivation of the ligand protein. The method provides a one step procedure to introduce both PEG and a metal chelator to proteins through a heterofunctional PEG precursor. The method eliminates the activation step and better controls the degree of protein modification. The procedure is a general one and can be broadly applied, for example, to the PEGylation of any protein, peptide or monoclonal antibody molecule that contains primary amino groups.

Specifically, a heterofunctional PEG molecule with one end of the polymer coupled to DTPA was designed as described in Example 3 but at the other end of the PEG molecule, a NH₂-reactive functional group isothiocyanate (SCN-) was introduced. This precursor molecule (SCN-PEG-DTPA) is obtained as lyophilized powder and can be stored at −20° C. for months.

By using introducing the SCN-group, PEGylation of proteins was subsequently achieved by simply mixing SCN-PEG-DTPA with protein in aqueous solution at 4° C. overnight without pre-activation of the proteins.

To demonstrate the utility of SCN-PEG-DTPA precursor in PEGylation and radiolabeling of proteins, annexin V was used as a model protein. Annexin V is a protein that binds with high affinity to phosphatidylserine exposed on the surface of apoptotic cells (cells undergoing programmed cell death). Using novel methods of the invention, annexin V was successfully conjugated to PEG-DTPA-¹¹¹In.

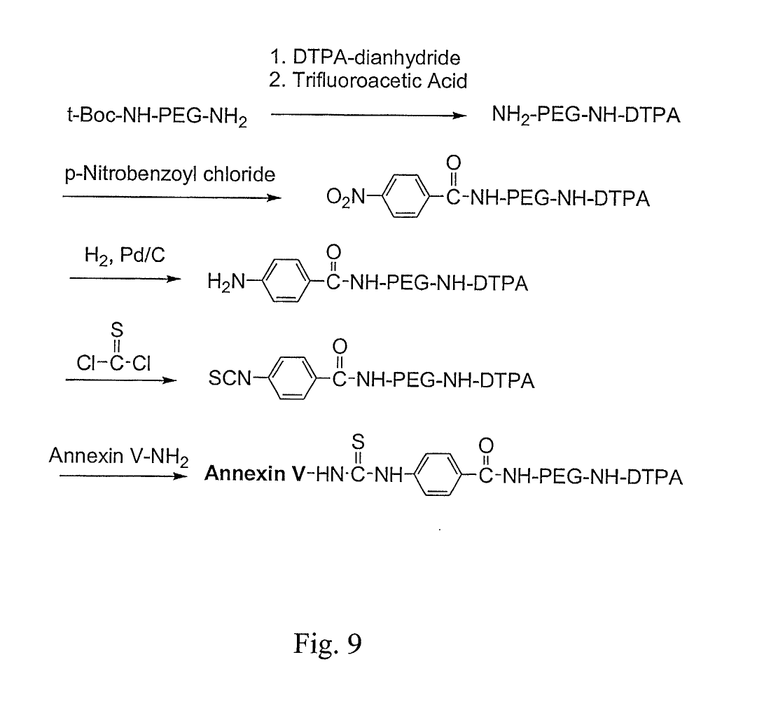

A preferred synthetic scheme for the preparation of the SCN-PEG-DTPA precursor is outlined in FIG. 9. DTPA was first coupled to mono-protected PEG diamine. After removal of the t-Boc protection group, the resulting NH₂-PEG-DTPA was reacted with p-nitrobenzoyl chloride, followed by catalytic hydrogenation to yield p-NH₂-benzoyl-PEG-DTPA. Treatment of p-NH₂-benzoyl-PEG-DTPA with thiophosgen yielded the target product SCN-PEG-DTPA with an overall yield of 74%.

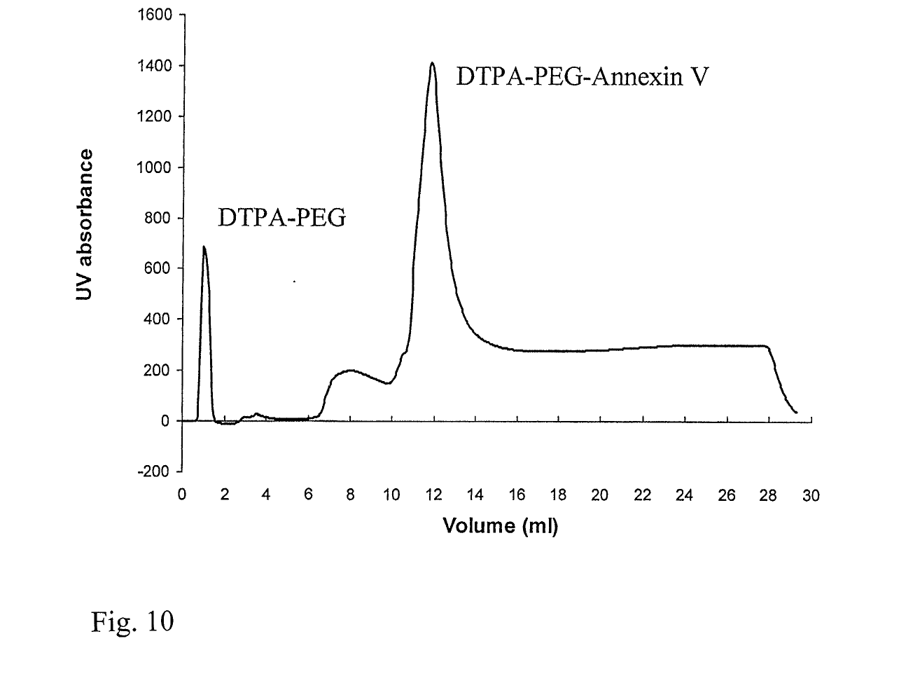

Annexin V was then conjugated to SCN-PEG-DTPA by simply mixing both agents together. The resulting conjugate was separated from unreacted PEG-DTPA by ion-exchange chromatography (FIG. 10). Since ion-exchange chromatography could not separate PEGylated annexin V from native annexin V, the reaction products were further analyzed by 15% sodium dodecyl sulfate-polyacrylamide gel electrophoresis (SDS-PAGE). DTPA-PEG-annexin V (DTPA-PEG-AV) was then radiolabeled with ¹¹¹In.

1:30 and 1:60 preps of PEGylated annexin V were tested for their ability to bind to cells that had been treated with Ara-C to induce apoptosis. To evaluate the binding of PEGylated annexin V to apoptotic cells, human leukemia HL60 cells and B-cell lymphoma Raji cells were treated with Ara-C at 1.0 μM for 22 hours. Cells were stained with annexin V-FITC and analyzed by flow cytometry. Alternatively, cells were incubated with the radiolabeled annexin V and cell associated radioactivity was measured. Flow cytometry revealed that the percentage of apoptotic cells increased 4-10 fold after treatment with Ara-C. Similarly, cell associated radioactivity with the 1:30 prep was also increased 4-6 fold.

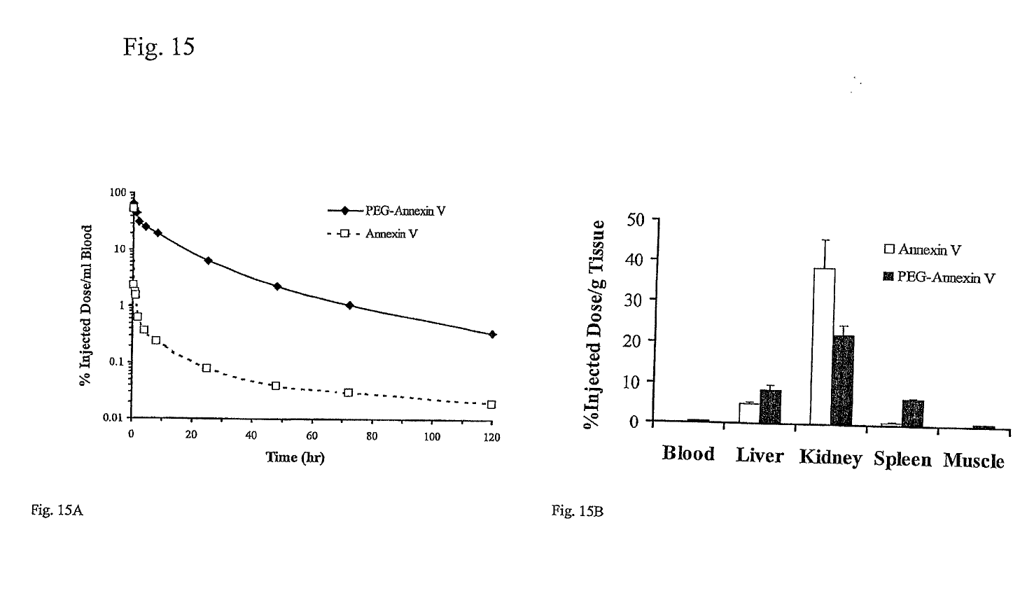

To demonstrate that PEGylation of annexin V improves the blood half life of the protein, the 1:15 prep of ¹¹¹In-DTPA-PEG-annexin V was injected into nude mice and blood was drawn at different time points after injection of the radiotracer. The annexin V activities in blood circulation after dosing with PEGylated annexin V were significantly higher than those from unPEGylated annexin V at all time points (FIG. 15A). Both profiles fit well into a two-compartment model and can be mathematically described by the equations of: Cₜ=41.0e.^{-0.14t}+8.0e^{-0.03t} for PEGylated annexin V and Cₜ=50.2e^{-9.51t}+0.33e^{-00.04t} for unPEGylated annexin V, where Cₜ is percentage injected dose per ml of blood at any given time, t. The half-life values from PEGylated annexin V were 4.90 hours and 26.3 hours for t_{1/2, α} and t_{1/2, β}, respectively, while those from unPEGylated annexin V were 0.07 hours and 17.4 hours for t_{1/2, α} and t_{1/2, β}, respectively. The modification with PEGylation significantly prolonged the terminal half-life from 17 hours with unPEGylated annexin V to 26 hours, resulting from a significant reduction in clearance, from 0.4 ml/hr with unPEGylated annexin V to 0.01 ml/hr. The PEGylated annexin V was less widely distributed in vivo, as reflected in the reduced volume distribution from 6.1 ml with unPEGylated annexin V to 0.2 ml. Biodistributions of 1:15 prep ¹¹¹In-DTPA-PEG-annexin V and ¹¹¹In-DTPA-annexin V at 120 hours after the injection of each radiotracer are summarized in FIG. 15B. PEGylation resulted in significantly reduced uptake of annexin V in the kidney and increased uptake in the liver and the spleen. For ¹¹¹In-DTPA-PEG-annexin V, the percentages of injected dose per gram of tissue for blood, liver, kidney, spleen, and muscle were 0.36±0.05%, 8.37±2.76%, 22.35±4.74%, 6.75±0.44, %, and 0.75±0.04%, respectively.

Finally, as shown in Examples 26 and 27, in vivo results demonstrate that apoptosis induced by PG-Paclitaxel correlates with the uptake of ¹¹¹In labeled PEGylated annexin V (¹¹¹In-DTPA-PEG-AV) in MDA-MB-468 tumors. This provides further support that ¹¹¹In-DTPA-PEG-AV and similar conjugates of the invention may be used to visualize apoptosis in cells, e.g., following chemotherapy.

Normally, native annexin V is rapidly cleared from the blood after intravenous injection. However, as demonstrated in the Examples, conjugation of PEG to annexin V according to the invention prolongs the circulation time of native annexin V. Because apoptosis is a dynamic process in which apoptotic cells are rapidly removed by phagocytic macrophages, longer or prolonged circulation of radiolabeled annexin V will make it possible to capture cells undergoing the early phase of apoptosis during a prolonged period of time. It will also make it possible to deliver radiolabeled annexin V to less perfused tissues. The prolonged blood half-life of preferred annexin V conjugates of the invention will allow more radiolabeled annexin V bind to apoptotic cells in tumor, resulting in improved imaging property.

Further, as shown in the examples, precursor PEG molecules were successfully synthesized and coupled to a ligand (annexin V) without having to preactivate the ligand. The demonstrated in vitro binding of the resulting conjugate to drug-treated cells, the favorable pharmacokinetics of ¹¹¹In-labeled, PEGylated annexin V, and the in vivo results indicate that radiolabeled, PEGylated annexin, which can be readily synthesized by the disclosed methods, may be used to image apoptosis and thus, to non-invasively image early responses to anticancer therapy.

Accordingly, one embodiment of the invention is directed to novel conjugate molecules which may be useful for the visualization and treatment of tumors or biological receptors, including visualization of response to therapy. The conjugates may be used in nuclear imaging, including radioimmunoscintigraphy and receptor-mediated imaging. A preferred embodiment is a conjugate molecule comprising a ligand (e.g., a protein, peptide or antibody), a polymer, a chelating agent, and a diagnostic agent, which is preferably a radioisotope. Preferably the ligand is bonded to the polymer, the chelating i5 agent is bonded to the polymer, and the radioisotope is chelated to the chelating agent. As used herein, “bonded” refers to any physical or chemical attachment, including, but not limited to, covalent bonding or ionic and chelating interactions. In a preferred embodiment, the ligand is covalently bonded to the polymer and the chelating agent is covalently bonded to the polymer.

The chelating agent can generally be any metal chelating agent, and most preferably is DTPA (diethylenetriamine pentaacetic acid). Other useful chelating agents include, but are not limited to: ethylenedicysteine (EC); dimercaptosuccinic acid (DMSA); ethylenediaminetetraacetic acid (EDTA); 1,2-cyclohexanediamine-N,N,N′,N′-tetraacetic acid (Cy-EDTA); ethylenediaminetetramethylenephosphonic acid (EDTMP); N-[2-[bis(carboxymethyl)amino]cyclohexyl]-N′-(carboxymethyl)-N,N′-ethylenediglycine (CyDTPA); N,N-bis[2-[bis(carboxymethyl)amino]cyclohexylglycine (Cy₂DTPA); 2,5,8-tris(carboxymethyl)-12-phenyl-11-oxa-2,5,8-triazadodecane-1,9-dicarboxylic acid (BOPTA); diethylenetriaminepentaacetic acid, monoamide (DTPA-MA); diethylenetriaminepentaacetic acid, biamide (DTPA-BA); diethylenetriamine-N,N,N′,N″,N″-pentamethylenephosphonic acid (DTPMP); tetraazacyclododecane-N,N′,N″,N′″-tetraacetic acid (DOTA); tetraazacyclotridecane-N,N′,N″,N′″-tetraacetic acid (TRITA); tetraazacyclotetradecane-N,N′,N″,N′″-tetraacetic acid (TETA); tetraazacyclododecane-α,α′,α″,α′″-tetramethyl-N,N′,N″,N′″-tetraacetic acid (DOTMA); tetraazacyclododecane-N,N′,N″,N′″-tetraacetic acid, monoamide (DOTA-MA); 10-(2-hydroxypropyl)-1,4,7,10-tetraazacyclododecane-1,4,7-triacetic acid (HP-DO3A); 1-((p-nitrophenyl)carboxymethyl)-4,7,10-tris(carboxymethyl)-1,4,7,10-tetraazacyclododecane (pNB-DOTA); tetraazacyclodecane-N,N′,N″,N′″-tekamethylenephosphonic acid (DOTP); tetraazacyclododecane-N,N′,N″,N′″-tetramethylenetetramethylphosphinic acid (DOTMP); tetraazacyclododecane-N,N′,N″,N′″-tetramethylenetetraethylphosphinic acid (DOTEP); tetraazacyclododecane-N,N′,N″,N′″-tetramethylenetetraphenylphosphinic acid (DOTPP); tetraazacyclododecane-N,N′,N″,N′″-tetramethylenetetrabenzylphosphinic acid (DOTBzP); tetraazacyclodecane-N,N′,N″,N′″-tetramethylenephosphonic acid-P,P′,P″,P′″-tetraethyl ester (DOTPME); hydroxyethylidenediphosphonate (HEDP); diethylenetriaminetetramethylenephosphonic acid (DTTP); N₃S triamidethiols; N₂S₂ diamidedithiols (DADS); N₂S₂ monoamidemonoaminedithiols (MAMA); N₂S₂ diaminedithiols (DADT); N₂S₄ diaminetetrathiols; N₂P₂ dithiol-bisphosphines; 6-hydrazinonicotinic acids; propylene amine oximes; tetraamines; and cyclams. For a general reference on metal chelating agents, see S. Liu et al., Bifunctional chelators for therapeutic lanthamide radiopharmaceuticals, Bioconjugate Chemistry 12: 7-34, 2001; see also S. S. Jurisson et al., Potential technitium small molecule radiopharmaceuticals, Chem. Rev. 99: 2205-2218, 1999; S. Liu et al., 99 mTc-Labeled small peptides as diagnostic radiopharmaceuticals, Chem. Rev. 99: 2235-2268, 1999.

Most preferably, the polymer is PEG. However, other polymers, particularly those which are biocompatible and water-soluble, may be used without departing from the scope of the invention. In addition to PEG, useful polymers include, but are not limited to, poly(1-glutamic acid), poly(d-glutamic acid), poly(d1-glutamic acid), poly(1-aspartic acid), poly(d-aspartic acid), poly(d1-aspartic acid), polylysine, a polysaccharide, dextran, polypropylene oxide (PPO), polyvinyl pyrolidone, polyvinyl alcohol, hyaluronic acid, chitosan, dextran, polyacrylic acid, poly(2-hydroxyethyl 1-glutamine) and carboxymethyl dextran. The polymer can also be a copolymer of two or more of the above polymers. Preferred polymers include polyethylene glycol, poly (1-glutamic acid), dextran, polyvinyl alcohol, polyethylene oxide-polypropylene oxide copolymer and copolymers between two or more of them.

The polymer can generally have any number average molecular weight, and preferably has a number average molecular weight of at least about 1,000 daltons. For example, the polyethylene glycol preferably has a number average molecular weight of about 1,000 daltons to about 100,000 daltons. The polysaccharide preferably has a number average molecular weight of about 1,000 daltons to about 150,000 daltons. The polyamino acid preferably has a number average molecular weight of about 1,000 daltons to about 150,000 daltons.

The ligand (or targeting moiety) can generally be any ligand, and preferably is an antibody or its fragments, a peptide or a protein. The antibody can generally be a monoclonal antibody, or a polyclonal antibody. For example, useful antibodies include, but are not limited to, C225, Herceptin, Rituxan, phage library antibodies, anti-CD, DC101, antibodies to the integrins alpha v-beta 3 (such as LM609), antibodies to VEGF receptors, antibodies to VEGF, or any other suitable antibody. The antibody can be an antibody fragment such as F(ab′)₂, Fab′, or ScFv fragment or an antibody fragment such as chimeric (c) 7E3Fab (c7E3Fab) that binds to integrin receptors. The antibody can be a humanized antibody. The peptide can generally be any peptide, such as a cell surface targeting peptide, and preferably is a growth factor, such as VEGF (Vascular Endothelial Growth Factor)-A, -B, -C, or -D, PDGF (Platelet-Derived Growth Factor), Angiopoietin-1 or -2, HGF (Hepatocyte growth Factor), EGF (Epidermal Growth Factor), bFGF (Basic Fibroblast Growth Factor), cyclic CTTHWGFTLC, cyclic CNGRC, or cyclic RGD-4C. The protein can generally be any protein, such as annexin V, interferons (e.g., interferon α, interferon β), tumor necrosis factors, endostatin, angiostatin, or thrombospondin, and preferably is annexin V, endostatin, angiostatin, or interferon-α. More preferably, the ligand is a monoclonal antibody, such as a C225, Herceptin or c7E3Fab antibody, or a protein, such as annexin V. Most preferably, the ligand is annexin V or C225. Preferably, the ligand has affinity for a target tissue. Preferred ligands bind specifically to receptors or other binding partners on the target tissue.

The diagnostic agent is preferably a metal ion. More preferably, the metal ion is a radioisotope. Preferably, the radioisotope is ¹¹¹In or ⁶⁴Cu. However, other isotopes may be used for diagnostic and therapeutic purposes, including, but not limited to, ⁶⁷Ga, ⁶⁸Ga, ⁸²Rb, ⁸⁶Y, ⁹⁰Y, ⁹⁹ mTc, ⁶⁷Cu, ¹⁹³pt, ^{113m}In, ²⁰¹Tl and other radiometals listed in Table I in C. J. Anderson, et al., Radiometal-Labeled Agents (Non-Technetium) for Diagnostic Imaging, Chem. Res. 99:2219-2234, 1999, incorporated herein by reference.

In one preferred embodiment, the ligand is annexin V. In a particularly preferred embodiment, the conjugate molecule comprises an annexin V-PEG-DTPA-¹¹¹In conjugate. In another preferred embodiment, the ligand is C225 and the conjugate molecule comprises a C225-PEG-DTPA-¹¹¹In conjugate. However, the invention is not limited to conjugates of these ligands, PEG, DTPA and ¹¹¹In. Rather, any suitable ligand, polymer, chelating agent, radioisotope or other diagnostic agent, including, but not limited to, those described herein, may be used in the conjugate without departing from the spirit and scope of the invention.

Although preferred conjugate molecules of the invention comprise at least one ligand and at least one chelating agent, multiple ligands and/or chelating agents can be present on a single conjugate molecule.

The invention also includes compositions comprising any of the above conjugate molecules and a pharmaceutically acceptable carrier. As used herein, “pharmaceutically acceptable carrier” includes any and all solvents, dispersion media, coatings, antibacterial and antifungal agents and isotonic agents and the like. The use of such media and agents for pharmaceutically active substances is well known in the art. For example, the carrier may comprise water, alcohol, saccharides, polysaccharides, drugs, sorbitol, stabilizers, colorants, antioxidants, buffers, or other materials commonly used in pharmaceutical compositions. Except insofar as any conventional media or agent is incompatible with the active ingredient, its use in the therapeutic compositions is contemplated. Supplementary active ingredients can also be incorporated into the compositions.

The phrase “pharmaceutically acceptable” also refers to molecular entities and compositions that do not produce an allergic or similar untoward reaction when administered to an animal or a human.

A preferred composition is a pharmaceutical preparation suitable for injectable use. Pharmaceutical preparations of the invention suitable for injectable use include sterile aqueous solutions or dispersions and sterile powders for the preparation of sterile injectable solutions or dispersions. Preferably, the preparations are stable under the conditions of manufacture and storage and are preserved against the contaminating action of microorganisms, such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms may be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Sterile injectable solutions may be prepared by incorporating the active compounds in the required amount in the appropriate solvent with various of the other ingredients enumerated above, as required, followed by filtered sterilization. Generally, dispersions may be prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation include vacuum-drying and freeze-drying techniques which yield a powder of the active ingredient plus any additional desired ingredient from a previously sterile-filtered solution thereof.

For parenteral administration in an aqueous solution, the solution is preferably suitably buffered, if necessary, and the liquid diluent first rendered isotonic with sufficient saline or glucose. These particular aqueous solutions are especially suitable for intravenous and intraperitoneal administration.

Another embodiment of the invention is directed to a method for synthesizing a ligand-polymer-chelating agent-diagnostic agent conjugate molecule comprising: providing an SCN-polymer-chelating agent precursor, wherein the polymer is preferably covalently bonded to the chelating agent and the SCN group is preferably covalently bonded to the polymer; combining a ligand with the SCN-polymer-chelating agent precursor to form a ligand-polymer-chelating agent conjugate; and combining the ligand-polymer-chelating agent conjugate with a diagnostic agent to form the ligand-polymer-chelating agent-diagnostic agent conjugate molecule. Preferably, the ligand comprises a primary amino group.

In the resulting conjugate molecule, preferably the ligand is covalently bonded to the polymer, the chelating agent is covalently bonded to the polymer, and the diagnostic agent is chelated to the chelating agent. The ligand may be covalently bound to the polymer, for example, by a thiourea, thioether, disulfide, iminourethane or urea bond, and preferably by a thiourea bond. The chelating agent may be attached to the polymer, for example, by an amide, thiourea or thioether bond, and preferably by an amide or thiourea bond. The ligand, polymer, chelating agent, and diagnostic agent may be any of the compounds mentioned herein. Preferably, the ligand is annexin V or C225, the polymer is PEG, the chelating agent is DTPA or DOTA, and the diagnostic agent is ¹¹¹In or ⁶⁴Cu.

Another embodiment is directed to a method for synthesizing a conjugate molecule comprising: providing a polymer conjugate-SCN precursor, wherein the SCN group is preferably covalently bonded to the polymer conjugate; and combining a ligand with the polymer conjugate-SCN precursor to form a ligand-polymer conjugate molecule, wherein the ligand is preferably covalently bonded to the polymer. Preferably, the ligand comprises a primary amino group.

The polymer conjugate may comprise a polymer bonded (e.g., via a covalent bond) to a chelating agent. Alternately, the polymer conjugate may comprise a polymer bonded (e.g., via a covalent bond) to any therapeutic agent. Alternately, the polymer conjugate may comprise a polymer bonded to another polymer, which in turn is bonded to a therapeutic agent, as more specifically described in U.S. Provisional Patent Application No. 60/334,969, entitled “Therapeutic Agent/Ligand Conjugate Compositions and Methods of Use,” filed Dec. 4, 2001, and incorporated herein by reference. As used herein “therapeutic agent” broadly includes, but is not limited to, drugs, chemotherapeutic drugs/agents, diagnostic agents (including radioisotopes and dye molecules), hormonal drugs/agents, and other compounds and compositions useful in the treatment and diagnosis of disease. Chemotherapeutic agents useful in the practice of the invention include, but are not limited to, Adriamycin (Adr), daunorubicin, paclitaxel (Taxol), docetaxel (taxotere), epothilone, camptothecin, cisplatin, carboplatin, etoposide, tenoposide, geldanamycin, methotrexate, maytansinoid DM1 or 5-FU. Other therapeutic agents that can be used include, but are not limited to, magnetic resonance imaging contrast agents such as gadolinium-DTPA (Gd-DTPA), and near-infrared optical imaging agents such as Cy 5.5, indocyanine green (ICG) and its derivatives, and Alexa fluor. However, the invention is not limited to the foregoing, and other compounds and agents may be used without departing from the scope of the invention.

In one preferred embodiment, the resulting conjugate molecule comprises a ligand, a polymer and a dye molecule, such as a near-infrared dye.

When the polymer conjugate comprises a polymer bonded to a chelating agent, the method may further comprise the step of combining the ligand-polymer conjugate molecule with a diagnostic agent to form a ligand-polymer-chelating agent-diagnostic agent conjugate molecule. Preferably, the diagnostic agent is a radioisotope. The radioisotope may be any of the radioisotopes described herein, and most preferably is ¹¹¹In or ⁶⁴Cu.

The ligand, chelating agent, polymers and therapeutic agents in the conjugates of the invention may be any of the compounds described herein. Preferably, the ligand is annexin V, the polymer is PEG and the chelating agent is DTPA or DOTA.

While certain preferred methods for synthesizing the conjugate molecules of the invention use isothiocyanate, other protein reactive reagents may be used in the practice of the invention. Useful protein reactive reagents include, for example, amine reactive and thiol reactive groups. In addition to isothiocyanates, useful amine reactive groups include, but are not limited to, N-hydroxy succinimidyl esters, isocyanates, p-nitrophenyl carbonates, benzotriazole carbonates, pentafluorophenyl carbonates, tresylates, aldehydes and epoxides. Useful thiol-reactive groups include, but are not limited to, maleimides, vinylsulfones and iodoacetamides.

The foregoing methods of synthesis do not require preactivation of the ligand. The invention also includes various methods for synthesizing conjugate molecules of the invention involving preactivation or other preparation of the ligand or polymer. One such method for synthesizing a conjugate molecule comprises the steps of: providing a polymer conjugate, wherein the polymer conjugate comprises at least one thio (SH) group covalently conjugated to the polymer conjugate; providing a ligand, the ligand comprising at least one thio reactive group; and combining the polymer conjugate and the ligand to form a ligand-polymer conjugate molecule, in which the ligand is preferably covalently bonded to the polymer by a thioether (S—C) bond. Preferably, the ligand is pretreated with an agent to introduce the thio-reactive group. Useful pretreating agents include, but are not limited to, vinyl sulfone or maleimide.

The step of providing the polymer conjugate may comprise the steps of: obtaining a precursor polymer conjugate having a protected thio group; and treating the precursor polymer with a deblocking agent to release a free thio group.

The polymer conjugate may comprise a polymer covalently bonded to a chelating agent. In this embodiment, the method may further comprise combining the ligand-polymer conjugate molecule with a diagnostic agent to form a conjugate molecule construct comprising a ligand-polymer-chelating agent-diagnostic agent construct. Preferably, in the construct, the ligand is covalently bonded to the polymer, the polymer is covalently bonded to the chelating agent, and the diagnostic agent is chelated to the chelating agent.

In an alternate embodiment, the polymer conjugate comprises a polymer covalently bonded to a diagnostic or other therapeutic agent.

The methods of the invention are not limited to processes where the ligand includes the thio reactive group. For example, the invention also includes a method for synthesizing a conjugate molecule comprising the steps of: providing a polymer conjugate and a ligand, wherein one of the polymer conjugate or the ligand comprises a thio group, and the other of the polymer conjugate or the ligand comprises a thio reactive group; and combining the polymer conjugate and the ligand to form a ligand-polymer conjugate molecule. The ligand is preferably covalently bonded to the polymer by a thioether (S—C) bond.

For example, in one embodiment, the thio group is attached to the ligand and the thio reactive group is attached to the polymer conjugate. The polymer conjugate may be prepared, for example, by attaching SPDP (N-succinimidyl 3-[2-pyridyldithio] proprionate (Pierce Chemical Co., Rockford, Ill.) or maleimide to the polymer conjugate.

In an alternate embodiment, the thio group is attached to the polymer conjugate and the thio reactive group is attached to the ligand. In this embodiment, the ligand is pretreated, for example, with maleimide or vinyl sulfone to introduce the thio reactive group.

The polymer conjugate may comprise a polymer bonded to a diagnostic or other therapeutic agent. Alternately, the polymer conjugate may comprises a polymer bonded to a chelating agent. In the latter case, the method may further comprise combining the ligand-polymer conjugate molecule with a diagnostic agent to form a conjugate molecule construct comprising a ligand-polymer-chelating agent-diagnostic agent construct. In the resulting conjugate molecule, preferably the ligand is covalently bonded to the polymer, the chelating agent is covalently bonded to the polymer, and the diagnostic agent is chelated to the chelating agent.

In the foregoing synthetic methods, the polymers, ligands, chelating agents and diagnostic agents may be any suitable component, including, but not limited to, any of the various components described herein.

The present invention also includes therapeutic and/or diagnostic applications using the above described conjugate molecules. Any of the conjugates described above where the radioisotope is ¹¹¹In, ⁶⁷Ga, ⁶⁸Ga, ⁸²Rb, ⁸⁶Y, ⁹⁰Y, ^{99m}Tc, ⁶⁴Cu, ⁶⁷Cu, ¹⁹³Pt, ¹¹³In ²⁰¹Tl or another radiotherapeutic isotope or diagnostic agent can be used in these therapeutic/diagnostic applications. One such embodiment is directed to a method for treating a patient having or suspected of having a tumor comprising the steps of administering a therapeutically effective amount of a conjugate molecule to the patient, wherein the conjugate molecule comprises a ligand bonded to a polymer, a chelating agent bonded to the polymer, and a radioisotope chelated to the chelating agent. Preferably, the ligand is covalently bonded to the polymer and the chelating agent is covalently bonded to the polymer. Preferably, the ligand has affinity for and selectively binds to the tumor. The ligand may be any ligand, and preferably is an antibody or protein such as Herceptin, C225 or annexin V. The radioisotope may be any radioisotope, but preferably is ⁹⁰Y, ⁶⁴Cu or ⁶⁷Cu. The chelating agent may be any chelating agent, but preferably is DTPA or DOTA. The dosage of the conjugate molecule can be increased or decreased to modulate the therapeutic effect on the targeted neoplasm.

As used herein the term “treating” a tumor is understood as including any medical management of a subject having a tumor. The term would encompass any inhibition of tumor growth or metastasis, or any attempt to visualize, inhibit, slow or abrogate tumor growth or metastasis. The method includes killing a cancer cell by non-apoptotic as well as apoptotic mechanisms of cell death.

As used herein, the term “tumor” includes benign and malignant tumors or neoplasia. In one embodiment, the tumor is a solid tumor, such as breast cancer, ovarian cancer, colon cancer, lung cancer, head and neck cancer, a brain tumor, liver cancer, pancreatic tumor, bone cancer, or prostate cancer. Alternately, the tumor may be a malignancy such as leukemia or lymphoma. In addition to tumors, the invention may be used to treat other conditions in which the ligand has an affinity for the target tissue being treated.

Another embodiment is directed to a method for selectively delivering a diagnostic agent to apoptotic cells in a patient comprising the steps of: administering a conjugate molecule to the patient having apoptotic cells, wherein the conjugate molecule comprises a ligand bonded to a polymer, wherein the ligand is annexin V, a chelating agent bonded to the polymer, and a radioisotope chelated to the chelating agent. Preferably, the ligand is covalently bonded to the polymer and the chelating agent is covalently bonded to the polymer. Because of the affinity of annexin V for apoptotic cells, the conjugate molecule is selectively delivered to these cells.

In a preferred method, the apoptotic cells are present following treatment of a target tissue, e.g., treatment of a tumor with a chemotherapeutic agent. The target tissue may be any desired tissue, including, but not limited to, a tumor or other neoplasm, inflammatory, infectious, reparative or regenerative tissue (including post trauma and post surgery tissues). The apoptotic cells in the target tissue may be a result of acute organ transplant rejection, hypoxic-ischaemic cerebral reperfusion injury, the toxic effects of chemotherapeutic agents to normal tissues, sickle cell disease, thalassemia, multiple sclerosis, rheumatoid arthritis and other diseases associated with an acutely increased rate of apoptosis.

A further embodiment of the invention is directed towards methods of visualizing tumors or biological receptors using any of the above described conjugate molecules and compositions. The methods may comprise administering a conjugate molecule to a patient having or suspected of having a tumor; and detecting the conjugate molecule. The conjugate molecule comprises a ligand bonded to a polymer, a chelating agent bonded to the polymer, and a radioisotope chelated to the chelating agent. Preferably, the ligand is covalently bonded to the polymer and the chelating agent is covalently bonded to the polymer. Preferably, the ligand has affinity for and selectively binds to the tumor. The detecting step may comprise detection of the radioisotope by radioscintigraphy, single photon emission computed tomography (SPECT), or positron emission tomography (PET). The uptake of the conjugate molecules can be detected and quantified. DOTA-⁶⁴Cu and DOTA-⁶⁷Cu conjugates are particularly useful for PET imaging and therapy.

The tumor can generally be any type of tumor, and more preferably, is a solid tumor, including any of those described herein. The ligand may be any ligand, but preferably is a protein or antibody, such as Herceptin, C225 or annexin V. The chelating agent may be any chelating agent, and preferably is DTPA or DOTA. The radioisotope may be any radioisotope, and preferably is ¹¹¹In or ⁶⁴Cu.

Still other embodiments are directed methods for visualizing apoptotic cells in tumors or other disease associated with an acutely increased rate of apoptosis, and in other tissues with biological receptors using any of the above described conjugate molecules and compositions. A preferred method for visualizing apoptotic cells in a patient comprises the steps of: administering a conjugate molecule to the patient having apoptotic cells, wherein the conjugate molecule comprises a ligand bonded to a polymer, wherein the ligand is annexin V, a chelating agent bonded to the polymer, and a radioisotope chelated to the chelating agent; and detecting the conjugate molecule. Preferably, the ligand is covalently bonded to the polymer and the chelating agent is covalently bonded to the polymer. The detecting step may comprise detection of the radioisotope by radioscintigraphy, SPECT, PET, MRI or near-infrared camera. A correlation between the detected isotopic signal and the presence or absence of the apoptotic cells can be calculated.

The apoptotic cells may be associated with one or more disease conditions, including, but not limited to, acute organ transplant rejection, inflammatory, infectious, reparative or regenerative tissue (including post trauma and post surgery tissues), hypoxic-ischaemic cerebral reperfusion injury, the toxic effects of chemotherapeutic agents to normal tissues, sickle cell disease, thalassemia, multiple sclerosis, rheumatoid arthritis, and other diseases associated with an acutely increased rate of apoptosis.

A similar method for visualizing tumors or apoptotic cells comprises the steps of administering a conjugate molecule to a patient suspected of having a tumor or apoptotic cells; and detecting the conjugate molecule. In this embodiment, the conjugate molecule comprises a ligand bonded to a polymer and a near-infrared dye bonded to the polymer. Preferably, near-infrared dye is ICG or an ICG derivative. The ligand has affinity for and selectively binds to the tumor or apoptotic cells. The detecting step may comprise detection of the near-infrared dye by a near-infrared camera.

In all of the foregoing therapeutic/diagnostic methods the patient can be any animal. Preferably the patient is a mammal. The mammal can be a human, a dog, a cat, a horse, a cow, a pig, a rat, a mouse or other mammal. More preferably, the patient is a human. As used herein, “patient” broadly includes, but is not limited to, a human or any animal being treated, tested or monitored in any kind of therapeutic, diagnostic, research, development or other application.

In the foregoing methods, the administering step may be performed parenterally, e.g., by intravascular, intraperitoneal, intramuscular or intratumoral injection. The conjugate molecule may be administered by inhalation or another suitable route. Preferably, administration is by intravascular injection.

In the foregoing therapeutic/diagnostic methods, a therapeutically effective amount of the conjugate molecules of the invention are preferably administered to achieve the desired effect (e.g., treatment of, delivery to, or visualization of, the target). The actual dosage amount of a composition comprising the conjugate molecules of the present invention administered to the patient to achieve the desired effect can be determined by physical and physiological factors such as body weight, severity of condition, the type of disease being treated/visualized, previous or concurrent therapeutic interventions, idiopathy of the patient and route of administration, as well as other factors known to those of skill in the art. The practitioner responsible for administration will, in any event, determine the concentration of active ingredient(s) in a composition and appropriate dose(s) for the individual subject.

The invention also includes kits incorporating the conjugate molecules of the invention. Any of the compositions described herein may be comprised in a kit. The kit may also contain means for delivering the formulation such as, for example, a syringe for systemic administration, an inhaler or other pressurized aerosol canister.

The kits may comprise a suitably aliquoted composition of the present invention. The components of the kits may be packaged either in aqueous media or in lyophilized form. The container means of the kits may include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the aerosol formulation, one or more components of an aerosol formulation, additional agents, and any other reagent containers in close confinement for commercial sale. Such containers may include injection or blow-molded plastic containers into which the desired vials are retained. The kit may have a single container, or it may have distinct container for each compound.

When the components of the kit are provided in one or more liquid solutions, the liquid solution is an aqueous solution, with a sterile aqueous solution being particularly preferred. However, the components of the kit may be provided as dried powder(s). When reagents and components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which a pharmaceutically acceptable formulation of the pharmaceutically composition, a component of an aerosol formulation and/or an additional agent formulation are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection or blow-molded plastic containers into which the desired vials are retained.

Irrespective of the number or type of containers, the kits of the invention may also comprise, or be packaged with, an instrument for assisting with the delivery of the aerosol formulation within the body of an animal. Such an instrument may be a syringe, an inhaler, air compressor or any such medically approved delivery vehicle.

The use of the above described conjugate molecules is advantageous over those previously described in the art. Preferred embodiments of the invention are useful for visualizing and treating tumors and other diseased tissues and, in the case of annexin V and similar conjugates, monitoring the response of tumors to therapy. The invention may be used in diagnostic, therapeutic, research and other applications. Preferred conjugates have improved in vivo half lives, exhibit reduced or eliminated accumulation in the liver, and may be adapted for therapeutic uses. The use of polymers reduces non-specific interaction with non-target tissues and reduces background activity. Attachment of the chelating agent to the polymer instead of to the ligand directly improves retention of the ligand's receptor binding affinity. The conjugate molecule design strategy is extremely flexible, and allows for the preparation of a wide array of molecules for different diagnostic and clinical uses. It allows both passive (when ligand is not attached) and active (when ligand is attached) targeting.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Materials

The following materials were used in Examples 1-16. Antibody C225 was kindly provided by ImClone Systems Inc. (New York, N.Y.). t-Boc-NH-PEG-NH₂ (MW 3,400) was obtained from Shearwater Polymers, Inc. (Huntsville, Ala.). N-succinimidyl s-acetylthioacetate (SATA), N-γ-maleimidobutyryloxysuccinimide ester (GMBS), 2,4,6-trinitrobenzenesulfonic acid (TNBS, 5% w/v aqueous solution), 5,5′-dithio-bis(2-nitrobenzoic acid) (Ellman's Reagent), sodium dodecyl sulfate (SDS), (3-(4,5-dimethylthiazol-2-yl)-2,5-diphenyltetrazolium bromide (MTT), and PBS (0.01 M phosphate buffered saline containing 138 nM NaCl, 2.7 nM KCl, pH 7.4) were purchased from Sigma Chemicals (St. Louis, Mo.). DTPA-dianhydride, trifluoroacetic acid (TFA, anhydrous), ninhydrin, triethylamine (TEA) and all the other solvents and reagents were purchased from Aldrich Chemicals Co. (St. Louis, Mo.). All chemicals and solvents were at least ACS grade and were used without further purification. ¹¹¹Indium radionuclide was obtained from Dupont-NEN (Boston, Mass.). PD-10 disposable column, Sephadex G-75 gel and Sephacryl S-200 high-resolution gel were purchased from Amersham Pharmacia, Biotech (Piscataway, N.J.). Spectra/Pro 6 dialysis tubing (molecular weight cut-off [MWCO] 2 KD) and Centricon-YM-10 centrifugal filter devices (MWCO 10 KD) were purchased from Fisher Scientific (Houston, Tex.). Silica gel 60 TLC plates were obtained from EM Sciences (Gibbstown, N.J.).

### Example 2

### Preparation of DTPA-C225

DTPA-C225 was prepared using a previously described method (Science 220: 613-615, 1983). Briefly, DTPA-dianhydride (4.6 mg, 12.8 μmol) was added to an aqueous solution of C225 (2.4 mg, 0.016 μmol; 2.4 mg/ml). For reaction efficiency, the pH of the reaction solution was kept at 7-8 by adding 0.1 M Na₂HPO₄. After incubation at room temperature for 1 hour, the solution was concentrated to half volume on a Centricon-YM 10 centrifugal filter and purified from free DTPA by gel filtration on a PD-10 column.

### Example 3

### Preparation of DTPA-PEG-NH₂

To a stirred suspension of DTPA-dianhydride (143 mg, 0.4 mmoles) in 4 ml chloroform was added TEA (81 mg, 0.8 mmoles) and t-Boc-NH-PEG-NH₂ (340 mg, 0.1 mmol). The mixture was allowed to react at room temperature for 2 hours. The reaction was followed by silica gel TLC using CHCl₃—MeOH (4:1 v/v) as the mobile phase; the plates were visualized by both iodine vapor and ninhydrin spray (0.1% ninhydrin solution in ethanol). TLC showed complete conversion of NH₂-PEG-NH-t-Boc (R_{f}=0.55, purple in ninhydrin) to DTPA-PEG-NH-t-Boc (R_{f}=0.4 with iodine vapor, negative in ninhydrin). After the reaction, the chloroform and TEA were removed under vacuum. The t-Boc protecting group was removed without purification by adding TFA (2 ml) to the resulting residue and stirring the mixture at room temperature for 4 hours. The resulting DTPA-PEG-NH₂ was purified by dialysis against PBS and deionized water using dialysis tubing (MWCO, 2 KD). R_{f}, 0.18 (chloroform-methanol; 4:1 v/v; ninhydrin spray); yield: 360 mg, 95%.

### Example 4

### Preparation of DTPA-PEG-ATA

DTPA-PEG-NH₂ (182 mg, 0.05 mmol) was reacted with SATA (14 mg, 0.06 mmol) in chloroform at room temperature for 1 hour, then purified by dialysis (MWCO, 2 KD) and by gel filtration on a PD-10 column to afford DTPA-PEG-ATA, R_{f}=0.27 (chloroform-methanol; 4:1 v/v; iodine vapor). ¹H NMR, 300 MHz, CDCl₃: 2.34 (s, 3H, CH₃COS—); 3.17-3.28 (m, 8H, —CH₂CH₂— in DTPA); 3.50 (s, 308H, —CH₂CH₂— in PEG with 77 repeating units); yield: 180 mg, 92%.

### Example 5

### Preparation of DTPA-PEG-C225

DTPA-PEG-ATA can be coupled to maleimide-activated antibodies following a simple in situ deprotection step to release the free SH group. Maleimide-activated C225 with different ratios of C225 to maleimide was prepared according to the following general procedure.

The general synthetic scheme for the preparation of DTPA-PEG-C225 is shown in FIG. 1. Other antibodies, polymers, and chelating agents can be easily substituted to arrive at alternative products.

To an aqueous solution of C225 (2.4 mg/ml; 4.8 mg, 0.032 pmol) at room temperature was added aliquots of GMBS in dimethylformamide (DW) (2.8 mg/ml). The mixture was stirred for 1 hour, then purified by gel filtration using a PD-10 column. Prior to conjugation with activated C225, the acetyl protecting group in DTPA-PEG-ATA was removed using hydroxylamine. For this purpose, an aliquot of NH₂OH (50 μl) in 0.1 M Na₂HPO₄ (0.5 M) was added to a solution of DTPA-PEG-ATA (7.7 mg, 1.92 μmoles) in 0.1 M Na₂HPO₄ (pH 8.5, 0.5 ml), then incubated at room temperature for 30 minutes. The resulting DTPA-PEG-SH containing free sulfhydryl group was then mixed with maleimide-activated C225 with DTPA-PEG-SH-to-maleimide molar ratio of 2:1 and incubated at 4° C. overnight. The final product was separated from unreacted DTPA-PEG by gel filtration on a Sephacryl S-200 column (1.5 cm×20 cm) with PBS as eluent. The presence of free sulfhydryl group was monitored using Elhman's agent.

Four DTPA-PEG-C225 conjugates with different degrees of C225 modification were synthesized. These conjugates were designated as 1:10, 1:20, 1:30, and 1:40 DTPA-PEG-C225, with the numbers being the molar ratios of antibody to GMBS in the maleimide-activating reaction. The physicochemical properties of the newly synthesized conjugates and some of the ¹¹¹In-labeled molecules are summarized in Table 1. Each C225 molecule contained approximately 50-60 free amino groups as measured by TNBS assay. In the 1:10, 1:20, 1:30 and 1:40 DTPA-PEG-C225 conjugates, approximately 20-25%, 40%, 60% and 70% of amino groups were substituted, respectively. DTPA-C225 with DTPA directly attached to C225 mAb was also synthesized for the purpose of comparison (Table 1). Because DTPA-anhydride was readily hydrolyzed in aqueous media, coupling of DTPA directly to C225 was an inefficient reaction. Only 10-20% of the amino groups in C225 were substituted by DTPA when the molar ratio of DTPA-dianhydride to C225 reached 800:1.

TABLE 1

| C225 | Molar ratio | NH₂ | Radiochemical | |
|---|---|---|---|---|
| conjugate | C225:GMBS | substitution | yield | Radiopurity |
| 1:10 | 1:10 | 20-25% | >70% | >97% |
| 1:20 | 1:20 | 40% | | |
| 1:30 | 1:30 | 60% | >70% | >99% |
| 1:40 | 1:40 | 70% | | |
| DTPA-C225 | 1:800^{a} | 10-20% | 40% | >99% |

^{a}Molar ratio of C225 to DTPA-dianhydride.

### Example 6

### Radiolabeling

Generally, 40 μg of each antibody conjugate in 100 μl PBS was incubated with 350-400 μCi of ¹¹¹InCl₃ (in 20 μl 1 M sodium acetate buffer, pH 5.5) at room temperature for 15 minutes. The resulting radioisotopic product was purified from free ¹¹¹In by gel filtration on a PD-10 column using PBS as the eluent. Fractions of 0.5 ml each were collected. The radioactivity of each fraction was measured by a radioisotope calibrator (Capintec Instruments, Ramsey, N.J.). The protein content in each fraction was determined using a Bio-Rad protein assay kit according to the manufacturer's instruction (Bio-Rad Laboratories, Hercules, Calif.). The fractions containing the protein were combined. The radiochemical yield, expressed as percentage of radioactivity of the protein fractions to the total loaded radioactivity, was calculated. The radiochemical purity was determined by gel permeation chromatography (GPC).

Two PEG-modified antibody conjugates, 1:10 DTPA-PEG-C225 and 1:30 DTPA-PEG-C225, as well as DTPA-C225 were radiolabeled with ¹¹¹In. The radiochemical yields of the two ¹¹¹In-DTPA-PEG-C225 conjugates were over 70%, whereas the yield of ¹¹¹In-DTPA-C225 was only 40%. The lower yield with ¹¹¹In-DTPA-C225 reflects the fact that a large amount of DTPA was introduced into the coupling reaction between DTPA-dianhydride and C225. Although extensive purification procedures including ultracentrifligation and gel filtration on GPC column were used, DTPA-C225 could still be contaminated by trace amounts of DTPA molecules, leading to low labeling efficiency.

### Example 7

### Determination of Degree of Modification

The degrees of substitution of C225 by maleimide was determined by quantifying the free amino groups remaining in the antibody using TNBS assay according to the published protocol (Bioconjugate Techniques, G. T. Hermanson, Ed., San Diego, Academic Press, pp. 112-114, 1996). Briefly, samples were dissolved in 0.1 M sodium bicarbonate (pH 8.5) at a concentration of 20-200 μg/ml. To 1 ml of each sample solution was added 0.5 ml TNBS solution in 0.1 M sodium bicarbonate (0.01%, w/v). After incubation at 37° C. for 2 hours, 0.5 ml of 10% SDS and 0.25 ml of 1 N HCl were sequentially added to each sample. The percentage of the reacted amino groups was determined by comparing the UV absorbance (335 nm) of the free amino groups in the modified antibody with that of those in the intact antibody.

### Example 8

### Gel Permeation Chromatograph

Analytical GPC was performed with a Waters HPLC system (Waters Corporation, Milford, Mass.) consisting of a 2410 refractive index detector and a 2487 dual λ UV detector applying a TSK-G3000 PW 7.5 mm×30 cm gel column (Tosoh Corporation, Japan). Samples were eluted with PBS containing 0.1% LiBr at a flow rate of 1 ml/min, and the products were detected by the refractive index and UV absorbance at 254 run. Radio-GPC was performed using an HPLC unit equipped with LDC pumps (Laboratory Data Control, Rivera Beach, Fla.), an LUDLUM radiometric detector (Measurement Inc, Sweetwater, Tex.), and an SP 8450 LTVNIS detector (Spectra-Physics, San Jose, Calif.). The samples were separated by a Phenomenex Biosep SEC-S3000 7.8 mm×30 cm column, eluted with PBS containing 0.1% LiBr at a flow rate of 1 ml/min, and detected by radioactivity and UV absorbance at 254 nm.

GPC was used to monitor the purity of C225 conjugates and ¹¹¹In-labeled C225 conjugates. Coupling of PEG to C225 increased the hydrodynamic volume of C225. The retention time of intact C225 (6.0 minutes) on TSK-G3000 column was shortened to 4.9 minutes for 1:30 DTPA-PEG-C225, suggesting that PEG molecules were chemically bound to the mAb. The GPC chromatogram of purified DTPA-PEG-C225 also indicated that gel filtration on Sephacryl S-200 column adequately removed unconjugated C225. However, when 1:30 DTPA-PEG-C225 was labeled with ¹¹¹In, it gave two peaks in radio-GPC, with retention times of about 5.9 minutes and 8.5 minutes, respectively. The major peak at 5.9 minutes corresponded to 1:30 ¹¹¹In-DTPA-PEG-C225 while the minor peak at 8.5 minutes, which reflects a retention time identical to that of ¹¹¹In-DTPA-PEG, was attributed to unconjugated DTPA-PEG. Thus, another gel filtration procedure was necessary to remove ¹¹¹In-DTPA-PEG. The 1:30 and 1:10 conjugates were eluted at 5.7 minutes and 6.1 minutes, respectively, reflecting shorter retention times than ¹¹¹In-DTPA-C225, 6.7 minutes. These results further confirmed that there were more PEG molecules attached to the 1:30 DTPA-PEG-C225 conjugate than to the 1:10 conjugate. The radiopurities of purified 1:30 ¹¹¹In-DTPA-PEG-C225, 1:10 ¹¹¹In-DTPA-PEG-C225, and ¹¹¹In-DTPA-C225 were >99%, >97%, and >99%, respectively (Table 1).

### Example 9

### Cell Lines

Human breast adenocarcinoma cell lines NMA-NO-468, MDA-NM-435, and human vulvar squamous carcinoma cell line A431 were obtained from Dr. Fan (The University of Texas M. D. Anderson Cancer Center, Houston, Tex.). The cells were maintained in 1:1 (v/v) Dulbecco's modified Eagle's medium (DMEM)/Ham's F-12 mixture supplemented with 10% fetal bovine serum (FBS) (Gibco Laboratories, Grand Island, N.Y.) at 37° C. in 5% CO₂/95% air. Both MDA-MB-468 and A431 cell lines express high levels of EGFR. To determine the EGFR expression in MDA-MB-435 cells, 80 μg of total protein from cell lysates of each sample were resolved by 10% SDS-PAGE. The proteins were electroblotted onto Immobilon-NC HAHY nitrocellulose membrane (Millipore Corporation, Bedford, Mass.). The membrane was blocked in 10% nonfat dry milk for 2 hours at room temperature, incubated with monoclonal anti-EGFR antibody (Sigma) at 4° C. overnight, and treated with horseradish peroxidase-conjugated goat anti-mouse secondary antibodies (Jackson ImmunoResearch Laboratories, Inc., West Grove, Pa.) for 1 hour at room temperature. A signal was detected using the ECL Western blotting detection system (Amersham Pharmacia Biotech).

### Example 10

### Competitive Binding Assays

MDA-MB-468 cells were seeded at 5×10⁷ cells/well onto 12-well plates in 10% FBS medium and allowed to attach overnight. The medium was replaced by DMEM/F-12 medium plus 0.2% bovine serum albumin (BSA), and 1 μg/ml of 1:30 ¹¹¹In-DTPA-PEG-C225 or ¹¹¹In-DTPA-C225 plus native C225 mAb at the indicated concentrations were added to the wells. After incubation at 37° C. for 2 hours, the cells were washed five times with PBS containing 0.2% BSA. The cells were then trypsinized and transferred to 5 ml disposable culture tubes. The level of radioactivity in each tube was measured with a Cobra Auto-gamma Counter (Packard Instrument Company, Downers Grove, Ill.).

Studies on cellular uptake of 1:30 ¹¹¹In-DTPA-PEG-C225 and ¹¹¹In-DTPA-C225 in MDA-MB-468 cells have shown that cell-associated radioactivity increased with increasing concentrations of radiolabeled C225.

A plateau was reached at 2 μg/ml for 1:30 ¹¹¹In-DTPA-PEG-C225. Therefore, a concentration of 1 μg/ml for the radiolabeled C225 was chosen for the competitive binding assay.

FIG. 2 shows the competitive binding of ¹¹¹In-DTPA-C225 (squares) and 1:30 ¹¹¹In-DTPA-PEG-C225 (diamonds) with native C225 to MDA-MB-468 cells. The cells were incubated with 1 μg/ml of each ¹¹¹In-labeled C225 conjugates plus unlabeled C225 at different concentrations. After incubation at 37° C. for 2 hours, the cell-associated radioactivity was measured with a gamma-counter. The data are expressed as counts per minute (CPM) as a percentage of control (v-axis) versus μg/ml unlabelled C225 α-axis) and presented as the means of triplicates with standard deviations.

The binding of both 1:30 ¹¹¹In-DTPA-PEG-C225 and ¹¹¹In-DTPA-C225 to MDA-MB-468 cells was displaced by C225 in a dose-dependent manner, suggesting that the binding is EGFR specific (FIG. 2). Furthermore, ¹¹¹In-DTPA-PEG-C225 was almost fully displaced by a 16-fold excess of C225, whereas ¹¹¹In-DTPA-C225 was only 80% displaced by a 16-fold excess of C225 and could not be fully displaced even by 40-fold excess of C225. The remaining 20% of cell-associated ¹¹¹In-DTPA-C225 represents nonspecific binding to the cells. These results suggest that modification of C225 with PEG reduced the nonspecific interaction of the antibody. When the contribution of non-specific binding is taken into consideration and is deducted from the cell-associated radioactivity, 44% of ¹¹¹In-DTPA-C225 and 33% of 1:30 ¹¹¹In-DTPA-PEG-C225 remained bound to the cells when the concentration of native C225 mAb was equal to the concentration of the labeled molecules (1 μg/ml). This means that ¹¹¹In-DTPA-C225 retained about 88% of the receptor binding affinity of native C225, while 1:30 ¹¹¹In-DTPA-PEG-C225 retained about 66% of the binding affinity.

### Example 11

### Immunoprecipitation and Western Blot Analysis

A431 cells were cultured with C225 or DTPA-PEG-C225 conjugates at 37° C. for 30 minutes, followed by washing the cells twice with cold PBS and lysis of the cells with a buffer containing 50 mM Tris-HCl, pH 7.4, 50 mM NaCl, 0.5% NP-40, 50 mM NaF, 1 mM Na₃PO₄, 1 mM phenylmethylsulfonyl fluoride, 25 μg/ml leupeptin, and 25 μg/ml aprotinin. The lysates were centrifuged at the fall speed of a microcentrifuge for 15 minutes and the supernatants were collected for protein concentration determination. Immunoprecipitation was performed by incubation of 100 μg of cell lysate with 40 μl Sepharose 4B-conjugated protein A at room temperature for 1 hour, followed by washing the immunoprecipitates three times with the lysing buffer and separation of immunoprecipitates with 7% polyacrylamide SDS-electrophoresis. Western blot was carried out by electronically transferring the samples into a nitrocellulose membrane and incubation of the membrane for 1 hour with an anti-EGFR antibody. The EGFR signals in the membrane were developed by the ECL chemoluminescence detection kit (Amersham, Arlington Heights, Ill.).

The ability of DTPA-PEG-C225 conjugates to bind to EGFR was investigated in A431 cells, which express a very high level of EGFR. The cells exposed to C225 or one of the three 1:10, 1:20, and 1:40 DTPA-PEG-C225 conjugates were lysed, followed by immunoprecipitation of the antibody-bound EGFR with protein A-Sepharose beads and visualization of the EGFR with Western blot analysis. All three DTPA-PEG-C225 conjugates with 20%, 50%, and up to 70% of amino groups in C225 substituted, retained their EGFR binding activities. However, the amounts of EGFR immunoprecipitated by C225 conjugates decreased with increasing degree of substitution, indicating that the binding affinity of the PEG-modified C225 decreased with the increasing number of PEG molecules attached to the mAb.

### Example 12

### MTT Assay

DiFi cells were seeded at 5×10⁴ cells/well onto 24-well culture plates. Cell viability after 72 hour treatment of the cells with C225 or DTPA-PEG-C225 was assayed by adding 50 μl of 10 mg/ml MTT (3-[4,5-dimethylthiazol-2-yl]-2,5-diphenyltetrazolium bromide) (Sigma) into 0.5 ml of culture medium and incubating the cells for 3 hours at 37 AC in a CO₂ incubator, followed by cell lysis with 500 μl of lysis buffer containing 20% SDS in dimethyl formamide/H₂O, pH 4.7, at 37° C. for more than 6 hours. An optical absorbance of cell lysate was determined by measuring the cell lysate at a wavelength of 595 nm and normalizing the value with the corresponding control of untreated cells.

It has previously been reported that blocking EGFR tyrosine kinase activity with C225 leads to cell cycle arrest and subsequent cell death through apoptosis in DiFi cells (Br. J. Cancer, 92:1991-1999, 2000). While the linker molecule PEG-DTPA itself had no effect on DiFi cell growth, all three conjugates, 1:10, 1:20, and 1:40 DTPA-PEG-C225, inhibited the tumor cell growth to the same extent as native C225, indicating that all conjugates were capable of inducing apoptosis in the DiFi human colon cancer cells.

### Example 13

### Pharmacokinetics

Nude mice (Harlan Sprague Dawley, Indianapolis, Ind.) were divided into two groups of 3 mice each and administered 1:30 ¹¹¹In-DTPA-PEG-C225 at a dose of 1.5 μg/mouse or ¹¹¹In-DTPA-C225 at a dose of 5 μg/mouse by intravascular injection. At predetermined intervals, blood samples (30-60 μl) were taken from the tail vein, and the radioactivity of each sample was measured with a gamma counter. The pharmacokinetic parameters for ¹¹¹In-DTPA-PEG-C225 and ¹¹¹In-DTPA-C225 were calculated from mean blood concentration values observed from the time of initial administration to 96 hours after administration using WinNonlin™ 2.1 software (Scientific Consulting, Inc., Lexington, Ky.).

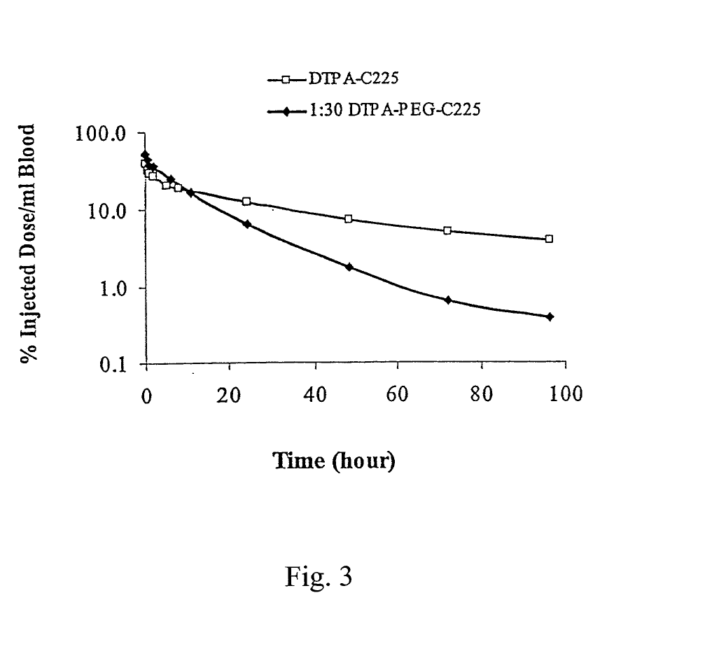

The radioactivity of the blood samples obtained at different time intervals after intravenous injection of 1:30 ¹¹¹In-DTPA-PEG-C225 and Lexington, Ky.). In-DTPA-C225 were measured with a gamma counter. FIG. 3 plots blood radioactivity following intravenous injection of radiolabeled C225.

Specifically, FIG. 3 depicts the pharmacokinetics of Lexington, Ky.). In-DTPA-C225 and 1:30 ¹¹¹In-DTPA-PEG-C225. The blood samples were collected at different time intervals, and the radioactivity of each sample was measured. The data are expressed as percentages of injected dose per ml of blood (% IND/ml) (v-axis) versus time in hours (x-axis) and presented as the means of triplicates. Squares indicate DTPA-C225; diamonds indicate 1:30 DTPA-PEG-C225. The standard derivation for each time point is less than a 10%.

The profiles of both ¹¹¹In-DTPA-PEG-C225 and ¹¹¹In-DTPA-C225 fit well into three-compartment models, and can be mathematically described by the tri-exponential equations, Cₜ=11.3 e^{-2.82t}+37.3 e^{-0.10t}+7.6 e^{-0.03t} and Cₜ=11.7 e^{-1-74t}+14.8 e^{-0.08t}+13.3 e^{-0.01t}, respectively, where Cₜ is % ID/ml blood at any given time t.

The pharmacokinetic parameters of volume distributions in the central compartment (V₁) and at steady state (Vₛₛ), clearance (CL), hybrid constants (A, B, C, α, β, γ), and microconstants (k₁₀, k₁₂, k₂₁, k₁₃, and k₃₁) are summarized in Table 2. The volume distribution at steady state (Vₛₛ) of 1:30 ¹¹¹In-DTPA-PEG-C225, 2.94 ml, was smaller than that of ¹¹¹In-DTPA-C225, 5.41 ml. The narrower distribution of PEG-modified molecules might be due to the reduced nonspecific binding of these molecules to tissues and the faster returning rate constant from tissues to the central compartment, reflected by the larger k₂₁. The elimination rate constant from the central compartment (k₁₀) and the clearance (CL) of 1:30 ¹¹¹In-DTPA-PEG-C225 were 0.09 h⁻¹ and 0.16 ml/h, respectively, higher than those of ¹¹¹In-DTPA-C225, 0.03 h⁻¹ and 0.08 ml/h, respectively. The terminal half-life (t_{1/2, γ}) of ¹¹¹In-DTPA-PEG-C225, 21.1 h, was shorter than that of ¹¹¹In-DTPA-C225, 52.9 hours, resulting from its smaller volume distribution and faster clearance.

TABLE 2

| Parameters | DTPA-C225 | DTPA-PEG-C225 (1:30) |
|---|---|---|
| A (% ID/ml) | 11.7 | 11.3 |
| B (% ID/ml) | 14.8 | 37.3 |
| C (% ID/ml) | 13.3 | 7.6 |
| α (h⁻¹) | 1.74 | 2.82 |
| β (h⁻¹) | 0.08 | 0.10 |
| γ (h⁻¹) | 0.01 | 0.03 |
| t_{½,α} (h) | 0.40 | 0.24 |
| t_{½,β} (h) | 9.11 | 6.82 |
| t_{½,γ} (h) | 52.9 | 21.1 |
| V₁ (ml) | 2.52 | 1.78 |
| Vₛₛ (ml) | 5.41 | 2.94 |
| CL (ml/h) | 0.08 | 0.16 |
| K₁₀ (h⁻¹) | 0.03 | 0.09 |
| K₁₂ (h⁻¹) | 0.48 | 0.53 |
| K₂₁ (h⁻¹) | 1.24 | 2.27 |
| K₁₃ (h⁻¹) | 0.03 | 0.02 |
| K₃₁ (h⁻¹) | 0.04 | 0.04 |

% ID/ml is the injected dose per ml of blood. A, B, C, α, β, and γ are hybrid constants. V₁ and Vₛₛ are volume distributions in the central compartment and at steady state (Vₛₛ). CL is clearance. K₁₀, K₁₂, K₂₁, K₁₃, and K₃₁ are microconstants.

Because the molecular weight of C225 is relatively high (150 KD), its modification with PEG (3.4 KD) was not expected to have profound effects on its blood circulation time. Nevertheless, this finding that the terminal half-life of 1:30 ¹¹¹In-DTPA-PEG-C225 was actually shorter than that of ¹¹¹In-DTPA-C225 is somewhat unexpected. A possible explanation is in vivo cleavage of ¹¹¹In-DTPA-PEG from ¹¹¹In-DTPA-PEG-C225. The shortened blood retention of ¹¹¹In-DTPA-PEG-C225 could be advantageous in obtaining improved images of target organs.

### Example 14

### Whole Body Scintigraphy and Dissection Analysis

Female BALB/c mice with a nu/nu background were subcutaneously injected with A431, MDA-MB-468, or MDA-MB-435 cells (1×10⁷/site) in the chest and the right hindlimb. When the xenografts reached 8-10 mm in diameter, the mice were divided into groups of 3 each. The mice were anesthetized by an intraperitoneal injection of sodium pentobarbital (35 mg/kg), then administered with 10 μg/mouse of 1:10 ¹¹¹In-DTPA-PEG-C225, 1:30 ¹¹¹In-DTPA-PEG-C225, or ¹¹¹In-DTPA-C225 (50-100 μCi) via tail vein. An Orbiter γ-camera (Siemens Gammasonics, Inc. Des Plaines, Ill.), equipped with a medium-energy collimator and Elscint Apex SPX-1 software, was used for the γ-imaging. The mice were placed prone on the camera's pinhole collimator with their heads pointing to the top. The images were acquired in a 128×128 matrix for 15 minutes, immediately after injection and at 5 minutes, 6, 24, and 48 hours after injection of radiotracer. Regions of interest were drawn on the computer images around the whole body, liver, muscle, and tumor. The counts per pixel in the tumor and normal tissues were calculated without correction for background level.

At 48 hours after injection, the mice were killed and dissected. Blood samples were obtained by cardiac puncture, and samples of the liver, muscle, and tumor were removed from each animal. Radioactivity of each sample was measured with the Cobra Auto-gamma Counter (Packard, Downers Grove, Ill.). The percentage of the injected dose per gram of tissue (% ID/g tissue) was calculated for each sample.

Whole-body gamma scintigrams of mice bearing A431 tumors obtained at different intervals after intravenous injection of ¹¹¹In-DTPA-C225, 1:10 ¹¹¹In-DTPA-PEG-C225, or 1:30 ¹¹¹In-DTPA-PEG-C225 are presented in FIGS. 4, 5, and 6, respectively.

FIG. 4 depicts sequential gamma images of a mouse injected intravenously with 10 μg ¹¹¹In-DTPA-C225. The mouse had A431 tumors (arrowhead) in the chest and right hindlimb. Whole body images were obtained 5 minutes and 6, 24, and 48 hours after injection. Radioactivity was predominantly in the liver (arrow) at 24 hours and 48 hours after injection. FIG. 5 depicts sequential gamma images of a mouse injected intravenously with 10 μg of 1:10 ¹¹¹In-DTPA-PEG-C225. Tumors are seen in the 24-hour postinjection image (arrowhead). FIG. 6 shows sequential gamma images of a mouse injected intravenously with 10 μg of 1:30 ¹¹¹In-DTPA-PEG-C225. Tumor (arrowhead) in the hindlimb is clearly seen at 6, 24, and 48 hours after injection.

Immediately after injection of each radiotracer, images showed the highest activity in the central location, which is attributable to the cardiac blood pool, the liver, and the spleen. While activity in the liver of mice injected with ¹¹¹In-DTPA-C225 dominated the images at 24 hours and 48 hours (FIG. 4), significant reduction of radioactivity in the liver was seen with PEG-modified C225 conjugates, particularly at 24 hours and 48 hours (FIGS. 5 and 6). Tumors at both inoculation sites (hindlimb and chest) were visualized 24 hours after injection with all three C225 radiotracers. However, only tumors in mice injected with 1:30 ¹¹¹In-DTPA-PEG-C225 were clearly seen 48 hours after injection (FIG. 6).

These observations were confirmed by image quantification. FIG. 7 shows the radioactivity in tumors expressed as tumor-to-whole body ratios per pixel obtained from sequential gamma camera images at the stated time intervals. All three radiotracers, demonstrated increased tumor radioactivity relative to whole body counts over time; this increase plateaued at 24 hours. The data in FIG. 7 are expressed as the ratios of tumor-to-whole body radioactivity per pixel (y-axis) versus time in hours (x-axis) and presented as the means ±SEM (n=3). Squares indicate ¹¹¹In-DTPA-C225; X's indicate 1:10 ¹¹¹In-DTPA-PEG-C225; and diamonds indicate 1:30 ¹¹¹In-DTPA-PEG-C225. No difference was found between mice that received PEG-modified C225 and those that received C225 without PEG modification (P>0.05).

FIG. 8 presents the tumor-to-liver ratios per pixel as a function of time. Specifically, FIG. 8 shows the quantification of tumor-to-liver ratios from sequential gamma images of ¹¹¹In-labeled C225 conjugates. The data are expressed as the ratios of tumor-to-liver radioactivity per pixel (y-axis) versus time in hours α-axis) and presented as the means ±SEM (n=3). Squares indicate ¹¹¹In-DTPA-C225; X's indicate 1:10 ¹¹¹In-DTPA-PEG-C225; and diamonds indicate 1:30 ¹¹¹In-DTPA-PEG-C225.

PEG-modified C225 conjugates had significantly higher tumor-to-liver ratios than C225 without PEG in modification at each analyzable time point (P<0.05), and the values increased with time until 24 hours after the radiotracer injection. Dissection analysis performed 48 hours after injection of the radiotracers showed that the liver uptake was markedly reduced in the mice that received PEG-modified radiotracer, from 46.9% ID/g for DTPA-C225 to 29.2% and 25.5% ID/g for 1:10 and 1:30 ¹¹¹In-DTPA-PEG-C225, respectively. The tumor uptake was unchanged with the lower degree of PEG modification (11.1% ID/g for 1:10 ¹¹¹In-DTPA-PEG-C225 vs. 11.0% ID/g for ¹¹¹In DTPA-C225) or decreased only moderately with the higher degree of PEG modification (8.7% ID/g for 1:30 ¹¹¹In-DTPA-PEG-C225).

### Example 15

### Effect of C225 Pretreatment on Imaging and Distribution

Mice with A431 tumors were pretreated with 1 mg of native C225 at 30 minutes or 20 hours before intravenous injection of 1:30 ¹¹¹In-DTPA-PEG-C225. Gamma scintigrams of the mice taken at 6 hours and 24 hours after the radiotracer injection showed markedly reduced liver uptake of ¹¹¹In-DTPA-PEG-C225 in both groups. While suppression of tumor activity was seen in mice injected with C225 20 hours before radiotracer injection, this effect was not obvious in scintigrams of mice given C225 30 minutes before injection of the radiotracer.

Dissection analysis performed 48 hours after injection of 1:30 ¹¹¹In-DTPA-PEG-C225 showed that pretreatment with C225 significantly reduced the tumor-to-blood ratio (P<0.05) (Tables 3 and 4; values represent mean ±standard deviation of 3 mice per group; data are expressed as percentages of injected dose per gram of tissue (% IND/g)). Tumor-to-muscle ratio also was reduced, albeit to a lesser degree. Pretreatment with C225 also significantly reduced liver uptake of 1:30 ¹¹¹In-DTPA-PEG-C225 (P<0.005 at 20-hour interval; P<0.05 at 30-minute interval). As a result, blood pool activity was significantly elevated. Notably, pretreatment with C225 only caused a moderate decrease in tumor uptake of the radiotracer when the interval between the administration of C225 and 1:30 was 20 hours. At a shorter interval (30 minutes) between the delivery of the two agents, the uptake of ¹¹¹In-DTPA-PEG-C225 in the tumor was actually significantly increased (P<0.05, Tables 2 and 3).

TABLE 3
Distribution of 1:30 ¹¹¹In-DTPA-PEG-C225

| Groups | Blood | Liver | Muscle |
|---|---|---|---|
| Radiotracer only | 1.40 ± 0.64 | 25.5 ± 2.0 | 0.69 ± 0.22 |
| C225 + radiotracer | 4.95 ± 0.61 | 16.5 ± 4.25 | 1.48 ± 0.22 |
| 30-min | | | |
| C225 + radiotracer | 2.93 ± 0.60 | 10.5 ± 0.40 | 0.73 ± 0.11 |
| 20-hour | | | |

TABLE 4
Distribution of 1:30 ¹¹¹In-DTPA-PEG-C225

| Groups | Tumor | Tumor/Blood | Tumor/Muscle |
|---|---|---|---|
| Radiotracer only | 8.68 ± 1.78 | 7.04 ± 1.94 | 13.4 ± 3.89 |
| C225 + radiotracer | 13.85 ± 1.38 | 2.83 ± 0.49 | 9.37 ± 0.58 |
| 30-min | | | |
| C225 + radiotracer | 7.63 ± 1.96 | 2.59 ± 0.14 | 10.4 ± 1.19 |
| 20-hour | | | |

### Example 16

### Imaging of Tumors as Function of EGFR Expression

To demonstrate that ¹¹¹In-DTPA-PEG-C225 can localize specifically in tumors that overexpress EGFR, human tumor xenografts expressing different levels of EGFR in the gamma imaging study were used. Western analysis of A431, MDA-MB-468, and MDA-MB-435 confirmed that both A431 and MDA-MB-435 express high levels of EGFR, while MDA-MB-435 express negligible amounts of EGFR. Gamma scintigrams of both A431 and MDA-MB-468 tumors in the chest and hindlimb sites demonstrated substantial activity 24 hours after injection of 1:30 ¹¹¹In-DTPA-PEG-C225. In contrast, less tumor activity was visualized in MDA-MB-435 tumors than in the other two tumor xenografts.

Forty-eight hours after injection of 1:30 ¹¹¹In-DTPA-PEG-C225, the tumor-to-blood ratios for A431 and MDA-MB-468 xenografts were significantly higher than those for the MDA-MB-435 xenografts (P=0.009 for A431 tumors; P<0.001 for MDA-MB-468 tumors). The ratios increased from about 1 for EGFR-negative MDA-MB-435 tumors to over 6 for EGFR-positive A431 and MDA-MB-468 tumors. Pretreatment with C225 20 hours before injection of 1:30 ¹¹¹In-DTPA-PEG-C225 significantly reduced the tumor-to-blood ratios for A431 (P=0.04) and MDA-MB-468 (P<0.001) tumors, but not for MDA-MB-435 tumors (P=0.3).

### Example 17

### Materials

The following materials were used in Examples 17-26. Annexin V (MW 33 kD, Lot 31k4055), annexin V-FITC, fluorescamine, PBS (0.01M phosphate buffered saline containing 138 nM NaCl, 2.7 nM KCl, pH 7.4), and poly(L-glutamic acid) (MW 31K) were purchased from Sigma Chemicals Co. (St. Louis, Mo.). t-Boc-NH-PEG-NH₂ (MW 3400) was obtained from Shearwater Polymers, Inc. (Huntsville, Ala.). Nitrobenzoyl chloride, triethylamine, palladium on activated carbon (10 wt. %), thiophosgen, ninhydrin, and all the other solvents and reagents were purchased from Aldrich Chemicals Co. (St. Louis, Mo.). Spectra/Pro 7 dialysis tubing (MWCO 2000) and Ultrafree centrifugal filter (MWCO 10,000) were purchased from Fisher Scientific (Houston, Tex.). Bio-Rad protein assay dye was purchased from Bio-Rad Laboratories (Hercules, Calif.). ¹¹¹In radionuclide was obtained from Dupont-NEN (Boston, Mass.). PD-10 disposable column was purchased from Amersham Pharmacia Biotech (Piscataway, N.J.). SDS-PAGE gel was purchased from Biowhittaker Molecular Applications (Rockland, Me.). Paclitaxel was obtained from Hande Tech (Houston, Tex.). Anti-EGFR monoclonal antibody C225 was generously provided by ImClone Systems, Inc. (New York, N.Y.). PG-TXL was synthesized according to previously reported procedures (C. Li, et al., Complete regression of well-established tumors using a novel water-soluble poly (L-glutamic acid)-paclitaxel conjugate. Cancer Res. 58:2404-2409, 1998).

¹H-NMR was recorded at 300 MHz on a Brucker Avance 300 spectrometer (Billerica, Mass.). Coupling constants are reported in hertz. FTIR was recorded on a Perkin-Elmer Spectrum GX system (Norwalk, Conn.). Purification of protein conjugates was accomplished using an AKTA FPLC system (Amersham Pharmacia Biotech, Piscataway, N.J.) equipped with a Resource Q 1 ml anionic ion exchange column (Amersham Pharmacia Biotech, Piscataway, N.J.). The samples were eluted with 20 mM Tris buffer (pH 7.5) and a linear gradient of 0-100% 1 N NaCl in 15 ml at a flow rate of 4 Ml/min. The products were detected by UV absorbance at 254 nm. Radio-gel permeation chromatography (GPC) was performed with a HPLC unit equipped with LDC pumps (Laboratory Data Control, Rivera Beach, Fla.), a LUDLUM radiometric detector (Measurement Inc., Sweetwater, Tex.), and an SP 8450 UV/VIS detector (Spectra-Physics, San Jose, Calif.). The samples were separated by a Phenomenex Biosep SEC-S3000 7.8 mm×30 cm column, eluted with PBS containing 0.1% LiBr at a flow rate of 1 ml/min, and detected by radioactivity and UV absorbance at 254 nm.

### Example 18

### Preparation of NH₂-PEG-DTPA

The PEG-DTPA derivative was prepared from t-Boc-NH-PEG-NH₂ (MW 3400) as described in Example 3 (See also, X. Wen, et al., Poly(ethylene glycol)-conjugated anti-EGF receptor antibody C225 with radiometal chelator attached to the termini of polymer chains, Bioconjugate Chemistry 12:545-553, 2001).

Specifically, to a stirred suspension of DTPA-dianhydride (143 mg, 0.4 mmoles) in 4 ml chloroform was added TEA (81 mg, 0.8 mmoles) and t-BocNH-PEG-NH₂ (340 mg, 0.1 mmol). The mixture was allowed to react at room temperature for 2 hours. The reaction was followed by silica gel TLC using CHCl₃—MeOH (4:1 v/v) as the mobile phase; the plates were visualized by both iodine vapor and ninhydrin spray (0.1% ninhydrin solution in ethanol). TLC showed complete conversion of NH₂-PEG-NH-t-Boc (R_{f}=0.55, purple in ninhydrin) to DTPA-PEG-NH-t-Boc (R_{f}=0.4 with iodine vapor, negative in ninhydrin). After the reaction, the chloroform and TEA were removed under vacuum. The t-Boc protecting group was removed without purification by adding TFA (2 ml) to the resulting residue and stirring the mixture at room temperature for 4 hours. The resulting DTPA-PEG-NH₂ was purified by dialysis against PBS and deionized water using dialysis tubing (MWCO, 2 KD). R_{f}, 0.18 (chloroform-methanol; 4:1 v/v; ninhydrin spray); yield: 360 mg, 95%.

### Example 19

### Preparation of p-NO₂-benzoyl-PEG-DTPA

NH₂-PEG-DTPA (0.07 mmoles, 280 mg) was reacted with p-nitrobenzoyl chloride (0.32 mmoles, 60 mg) in chloroform in the presence of triethylamine to afford p-nitrobenzoyl-PEG-DTPA. The reaction was complete after 4 hours shown by ninhydrin test. The resulting compound was purified by dialysis against PBS and deionized water using MWCO 2000 dialysis tubing. Yield 90%. ¹H NMR 300 MHz (D₂O), δ 8.36 ppm (d, J=8.7, 2H, φ-H), 7.98 ppm (d, J=8.7, 2H, φ-H), 3.4-3.9 ppm (m, 320H, —CH₂CH₂— in PEG and DTPA).

### Example 20

### Preparation of p-NH₂-benzoyl-PEG-DTPA

p-NO₂—PEG-DTPA (0.0325 mmoles, 130 mg) was dissolved in 20 ml water (pH 11, adjusted with 1 N NaOH) containing 25 mg of 10% Pd/C. The mixture was shaken overnight under 35 psi H₂, using hydrogenation apparatus (Parr Instrument Company, Moline, Ill.). The product was positive to fluorescamine test, showing green fluorescence under 366 nm UV light when sprayed with a solution of fluorescamine in acetone (0.05%, w/v). The reaction solution was neutralized with 1 N HCl, filtered to remove the catalyst, dialyzed against water (MWCO 2000), and lyophilized to give 118 mg (91%) white powder. ¹H NMR 300 MHz (D₂O), δ 7.65 ppm (d, J=8.6, 2H, φ-H), 6.85 ppm (d, J=8.6, 2H, φ-H), 3.4-3.9 ppm (m, 320H, —CH₂CH₂— in PEG and DTPA).

### Example 21

### Preparation of p-SCN-benzoyl-PEG-DTPA (SCN-PEG-DTPA)

p-NH₂-benzoyl-PEG-DTPA (0.03 mmoles, 120 mg) was reacted with 0.3 mmoles of thiophosgen in chloroform. The reaction was complete in 2 hours at room temperature as demonstrated by fluorescamine spray, which showed the disappearance of aromatic amine in p-NH₂-benzoyl-PEG-DTPA as the reaction proceeded. The solvent and excess of thiophosgen was removed under vacuum. Five ml of chloroform was added into the reaction vessel and removed under vacuum to remove residual thiophosgen. Yield 95%. FTIR, νₘₐₓ 2102 cm⁻¹ (SCN— stretch). ¹H NMR 300 MHz (D₂O), δ 7.83 ppm (d, J=8.6, 2H, φ-H), 7.44 ppm (d, J=8.6, 2H, φ-H), 3.4-3.9 ppm (m, 320H, —CH₂CH₂— in PEG and DTPA).

### Example 22

### Preparation of DTPA-PEG-Annexin V (DTPA-PEG-AV)

To a 0.5 mg/ml solution of annexin V (MW 33 kD, 0.2 mg, 0.006 μmole) in 0.1M Na₂HPO₄ (pH 8.5) was added aliquots of 0.18 μmoles (30 equivalents) or 0.36 μmoles (60 equivalents) of SCN-PEG-DTPA in dimethylformaldehyde (DMF) (36 mg/ml). The mixture was stirred at 4° C. overnight.

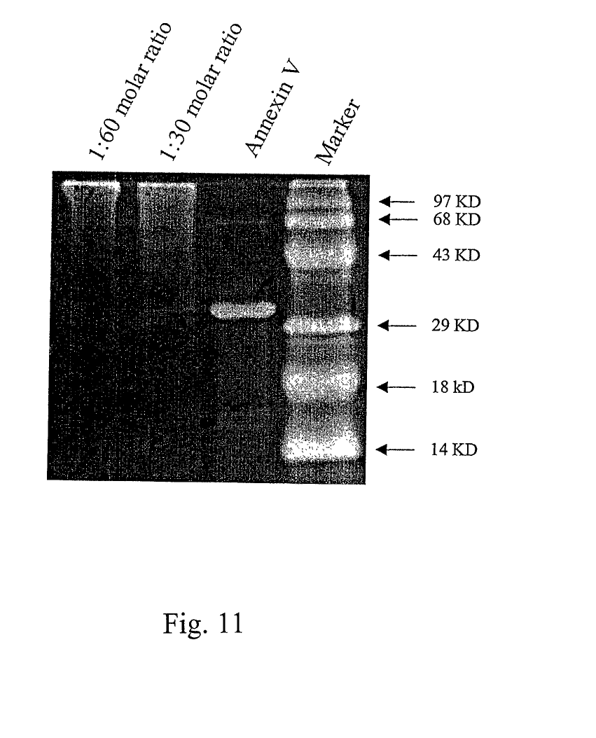

The products were separated from unreacted SCN-PEG-DTPA using an AKTA FPLC system (Amersham Pharmacia Biotech, Piscataway, N.J.) equipped with a Resource Q 1 ml anionic ion exchange column (Amersham Pharmacia Biotech). (FIG. 10—Purification of DTPA-PEG-Annexin V by ion exchange chromatography.) The samples were eluted with 20 mM Tris buffer (pH 7.5) and a linear gradient of 0-100% 1 N NaCl in 15 ml at a flow rate of 4 ml/min. The products were detected by UV absorbance at 254 nm. Fractions of 1 ml each were collected, and those that contained proteins (positive to Bio-Rad protein assay kit) were pooled and concentrated on Ultrafree centrifugal filter (MWCO 10,000). The purified products were analyzed for their purity by 15% acrylamide SDS-PAGE gel. (FIG. 11—SDS-PAGE of isolated products from the reaction between annexin V and SCN-PEG-DTPA at corresponding molar ratios of 1:60 and 1:30, respectively.)

The conversion of annexin V to its PEG conjugates was demonstrated by the disappearance of the band attributable to annexin V(˜33 K) and appearance of bands at higher molecular weights (FIG. 11). At a molar ratio of annexin V to SCN-PEG-DTPA of 1:60, all annexin V was PEGylated. At a molar ratio of 1:30, only a trace amount of unmodified annexin V was still present (FIG. 11).

### Example 23

### Radiolabeling of DTPA-PEG-AV

1:30 and 1:60 preps of DTPA-PEG-AV were labeled with Indium-111 (¹¹¹In) as described (X. Wen, et al., Poly(ethylene glycol)-conjugated anti-EGF receptor antibody C225 with radiometal chelator attached to the termini of polymer chains, Bioconjugate Chemistry 12:545-553, 2001). Briefly, 60 μg of DTPA-PEG-annexin V in 0.3 ml of 20 mM Tris buffer (pH 7.5) was incubated with 800 μCi of ¹¹¹InCl₃ in sodium acetate buffer (pH 5.5) for 15 minutes. Free ¹¹¹In was removed by gel filtration on PD-10 column using PBS as eluent. The purity of the radiolabeled annexin V was analyzed by radio-gel permeation chromatography. Radiochemical purity>98%; radiochemical yield=91%.

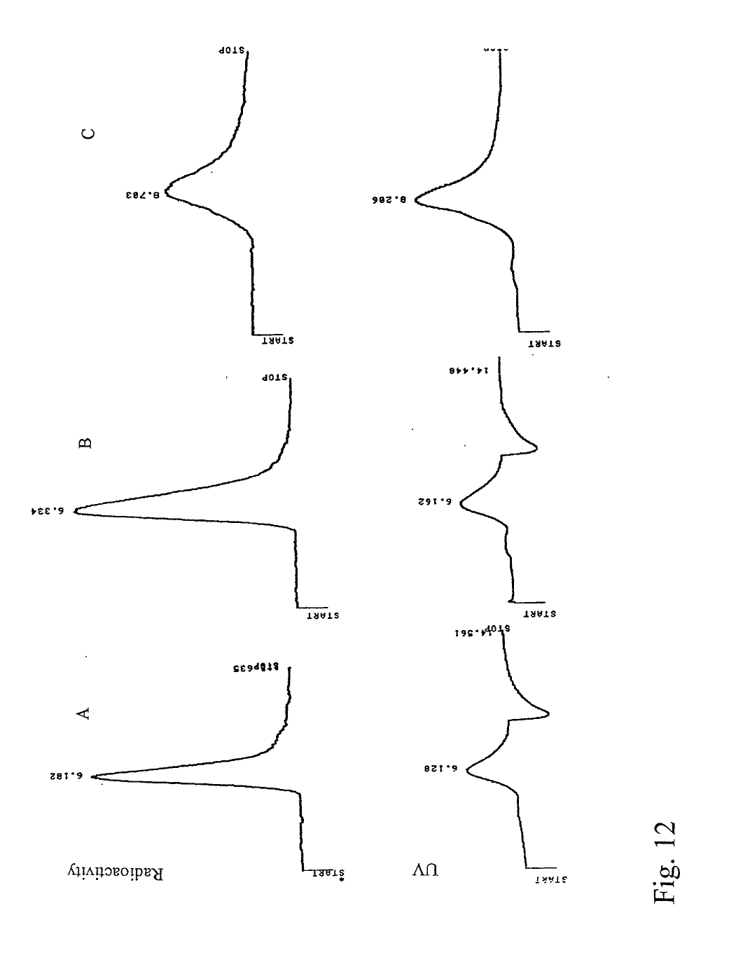

The three products were eluted through a Phenomenex Biosep SEC-S3000 column at 6.1, 6.3 and 8.7 min, respectively at a flow rate of 1 ml/min (FIG. 12—Radio-gel permeation chromatography of (A) 1:60 prep ¹¹¹In-DTPA-PEG-annexin V, (B) 1:30 prep ¹¹¹In-DTPA-PEG-annexin V, and (C) ¹¹¹In-DTPA-PEG).

GPC chromatograms of both 1:60 and 1:30 preps of ¹¹¹In-DTPA-PEG-AV revealed both radioactivity peaks and corresponding UV absorbance peaks at the same retention times of 6.1 and 6.3 minutes, respectively, indicating successful labeling of annexin V with ¹¹¹In (FIG. 12). No free ¹¹¹In-DTPA-PEG (retention time 8.7 minutes) was detected in both preps.

### Example 24

### Cell Binding Assay

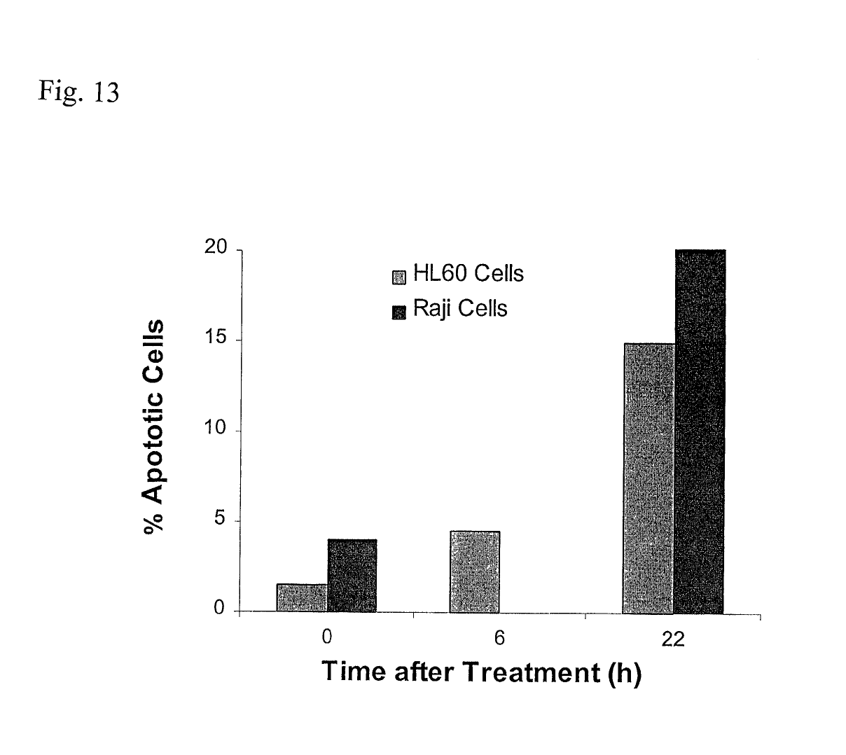

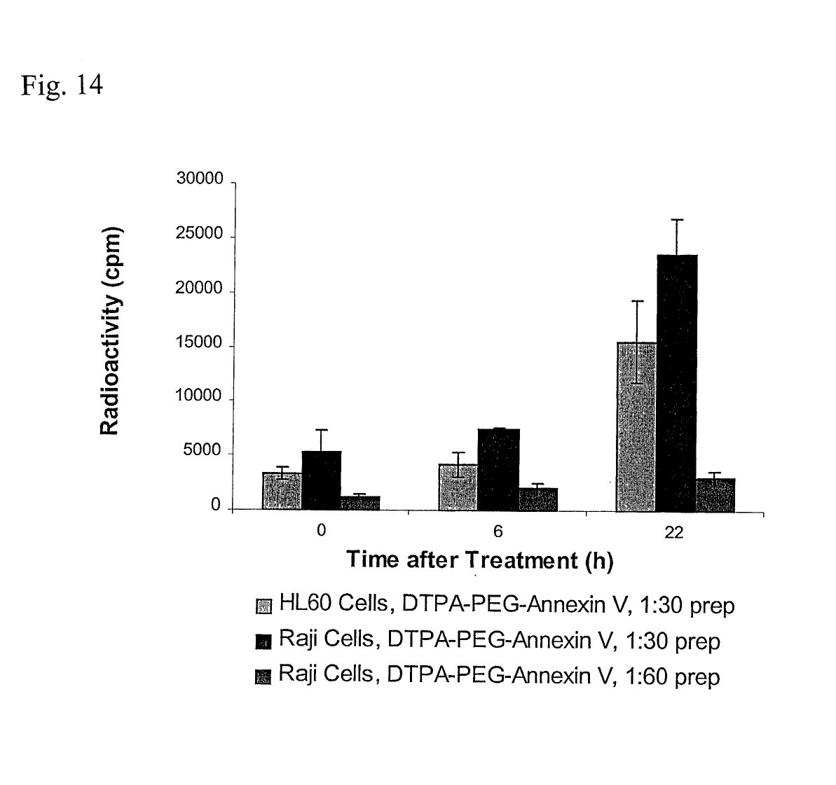

The 1:30 and 1:60 preps of PEGylated annexin V were tested for their ability to bind to cells that had been treated with Ara-C to induce apoptosis. Human leukemia HL60 or human B-cell lymphoma Raji cells (1×10⁶ cells/ml each) were treated with Ara-C at 1.0 μM for 6 hours or 22 hours to induce apoptosis. The cells were then washed twice with PBS and re-suspended in binding buffer (10 mM HEPES/NaOH, pH 7.5 containing 140 mM NaCl and 2.5 mM CaCl₂) at a concentration of 1×10⁶ cells/ml. Five gl annexin V-FITC solution (50 μg/ml in 50 mM Tris-HCl, pH 7.5, containing 100 mM NaCl) was added into the control cells as well as treated cells. The mixtures were incubated at room temperature for 10 minutes. Apoptotic cells stained with annexin V-FITC (Sigmna) were quantified with a Labsystems Fluoroskan FL flow cytometer (Helsinki, Finland) (FIG. 13). Alternatively, 1×10⁶ cells were suspended in 0.3 ml of binding buffer and incubated with 30 ¹¹¹In-DTPA-PEG-AV (330 μCi/ml, 33 μg/ml) at room temperature for 30 min. The cells were washed twice with PBS, centrifuged and counted for cell-associated radioactivity (FIG. 14).

FIG. 13 is a bar graph showing apoptotic index after treatment with 1.0 uM Ara-C as quantified by flow cytometry analysis using annexin V-FITC as fluorescent probe. In FIG. 13, the y-axis represents the percent apoptotic cells; the x-axis represents the time after treatment in hours. In the paired bars, the bars on the left represent HL60 cells. The bars on the right represent Raji cells. FIG. 14 is a bar graph showing binding of ¹¹¹In-DTPA-PEG-annexin V to Ara-C treated cells. In FIG. 14, the y-axis represents radioactivity in cpm; the x-axis represents time after treatment in hours. In each group of bars, the leftmost bars represent HL60 cells, DTPA-PEG-AV, 1:30 prep; the middle bars represent Raji cells, DTPA-PEG-AV, 1:30 prep; and the rightmost bars represent Raji cells, DTPA-PEG-AV, 1:60 prep.

As shown in FIG. 13, flow cytometry analysis using fluorescent annexin V-FITC as a probe showed that treatment with Ara-C at a concentration of 1.0 μM induced apoptosis of both HL60 cells and Raji cells, with increasing percentage of cells undergoing apoptotic process as the time of treatment increased (FIG. 13). As shown in FIG. 14, a similar trend was observed with 1:30 prep of ¹¹¹In-DTPA-PEG-AV. The radioactivity associated with cells treated with Ara-C increased with increasing time (FIG. 14). Specifically, flow cytometry revealed that the percentage of apoptotic cells increased 4-10 fold after treatment with Ara-C. Similarly, cell associated radioactivity was also increased 4-6 fold. Interestingly, the 1:60 prep of ¹¹¹In-DTPA-PEG-AV did not show binding to the Ara-C treated cells, suggesting that extensive modification abolished the binding affinity of annexin V to phosphatidylserine. These results suggest that the 1:30 prep binds to apoptotic cells the same way as annexin V-FITC does, and that the 1:30 prep is more suitable for imaging of apoptotic cells.

### Example 25

### Pharmacokinetics and Biodistribution

Nude mice (Harlan Sprague Dawley, Indianapolis, Ind.) were divided into three groups of 3 mice each and a 1:15 prep of ¹¹¹In-DTPA-PEG-annexin V, or ¹¹¹In-DTPA-annexin V was administered i.v. at a dose of 7 μg /mouse (70 μCi/mouse) to each group of mice. At predetermined intervals, blood samples (30-60 μl) were taken from the medial saphenous vein by puncture, and the radioactivity of each sample was measured with a Cobra Autogamma counter (Packard, Downers Grove, Ill.). The pharmacokinetic parameters for each radiotracer were calculated from mean blood concentration values observed over the study period using WinNonlin™ 2.1 software (Scientific Consulting, Inc., Lexington, Ky.). The mice were killed at the end of the study, and the liver, muscle, kidneys, and spleen were removed, weighed, and measured for radioactivity. The data are expressed as percentage of injected dose per gram of tissue.

The activity-time curves of ¹¹¹In-DTPA-PEG-AV and ¹¹¹In-DTPA-AV are presented in FIG. 15A and their tissue distributions are presented in FIG. 15B.

In FIG. 15A, the y-axis represents % Injected dose/ml blood; the x-axis represents time in hours. Squares represent Annexin V. Diamonds represent PEG-Annexin V. In FIG. 15B, the y-axis represents % Injected dose/g tissue. From left to right, the paired bars in FIG. 15B represent blood, liver, kidney, spleen, and muscle tissues, respectively. The bars on the left of the paired bars represent Annexin V. The bars on the right of the paired bars represent PEG-Annexin V.

The annexin V activities in blood circulation after dosing with PEGylated annexin V were significantly higher than those from unPEGylated annexin V at all time points (FIG. 15A). Both profiles fit well into a two-compartment model and can be mathematically described by the equations of: Cₜ=41.0e^{-0.14t}+8.0e^{-0.03t} for PEGylated annexin V and Cₜ=50.2e^{-9.51t}+0.33e^{-0.04t} for unPEGylated annexin V, where Cₜ is percentage injected dose per ml of blood at any given time, t. The half-life values from PEGylated annexin V were 4.90 hours and 26.3 hours for t_{1/2, α} and t_{1/2, β}, respectively, while those from unPEGylated annexin V were 0.07 hours and 17.4 hours for t_{1/2, α} and t_{1/2, β}, respectively. Biodistributions of 1:15 prep ¹¹¹In-DTPA-PEG-annexin V and 111In-DTPA-annexin V at 120 hours after the injection of each radiotracer are summarized in FIG. 15B. PEGylation resulted in significantly reduced uptake of annexin V in the kidney and increased uptake in the liver and the spleen. For ¹¹¹In-DTPA-PEG-annexin V, the percentages of injected dose per gram of tissue for blood, liver, kidney, spleen, and muscle were 0.36±0.05%, 8.37±2.76%, 22.35±4.74%, 6.75±0.44, %, and 0.75±0.04%, respectively.

### Example 26

### Apoptosis Induced by PG-Paclitaxel Correlates with Uptake of ¹¹¹In Labeled PEGylated Annexin V in MDA-MB-468 Tumors

The following materials and methods were used in this example. Human breast adenocarcinoma MDA-MB-468 cells were maintained in 1:1 (v/v) Dulbecco's modified Eagle's medium (DMEM)/Ham's F-12 mixture supplemented with 10% fetal bovine serum (FBS) (Gibco Laboratories, Grand Island, N.Y.) at 37C in 5% CO2/95% air. Female BALB/c mice with a nu/nu background were subcutaneously injected with MDA-MB-468 (1×10⁷ cells/site) in the chest. When the xenografts reached 6-8 mm in diameter, the mice were divided into groups of 4 each. Mice in Group I were not treated and were used as a control. In Group II, mice were injected intravenous with PG-paclitaxel (PG-TXL) at an equivalent paclitaxel (Taxol, TXL) dose of 100 mg/kg 1 day before the injection of radiotracer. PG-TXL is a water-soluble polymeric conjugate of paclitaxel and poly(1-glutamic acid). It has demonstrated significant antitumor activity with reduced systemic toxicity in preclinical and clinical studies. The conjugate has the same mechanism of action as paclitaxel. Both paclitaxel and PG-TXL block cells in the G2/M phase of cell cycle and subsequently induce apoptosis. In Group III, mice were injected with PG-TXL at an equivalent dose of 100 mg/kg 4 days before the injection of radiotracer. In Group IV, mice were injected i.p. with C225 at a dose of 50 mg/kg 4 days before the injection of radiotracer. C225 is a monoclonal antibody against EGFR. In Group V, mice were treated intravenous with PG-TXL (100 mg eq./kg) and i.p. with C225 (50 mg/kg) simultaneously 4 days before the injection of the radiotracer.

For imaging studies, the mice were anesthetized by an intraperitoneal injection of sodium pentobarbital (35 mg/kg), then administered with 8 μg/mouse of 1:30 prep of ¹¹¹In-labeled, PEGylated annexin V (50 μCi/mouse) via tail vein. A DigiRad camera (Model 2020tc, San Diego, Calif.) equipped with a medium-energy collimator and Mirage processing software (Segami Corp) was used for the gamma-imaging. The mice were placed prone on the camera's parallel hole collimator. The images were acquired in a 64×64 matrix for 5 minutes, at 2, 24, and 48 hours after injection of radiotracer.

Immediately after the last imaging session, the mice were killed and dissected. Blood samples were obtained by cardiac puncture, and samples of the liver, kidney, spleen, muscle, and tumor were removed from each animal. Radioactivity of each sample was measured with the Cobra Auto-gamma Counter (Packard, Downers Grove, Ill.). The percentage of the injected dose per gram of tissue (% ID/g tissue) was calculated for each sample.

In a separate experiment, tumors were histologically analyzed to quantify apoptotic index induced by drug treatments. Groups of mice were treated with PG-TXL (100 mg eq./kg), C225 (50 mg/kg), or combination of PG-TXL (100 mg eq./kg) and C225 (50 mg/kg), respectively. Mice were killed at day 1 and day 4 after treatments, and the tumors were immediately excised and placed in neutral-buffered formalin. The tissues were then processed and stained with hematoxylin and eosin. Apoptosis index was scored in coded slides by microscopic examination at 40× magnification. Five fields of normecrotic areas were randomly selected in each histological specimen, and in each field the number of apoptotic nuclei were recorded as numbers per 100 nuclei and were expressed as a percentage. The values were based on scoring 1500 nuclei obtained from 3 mice per time point.

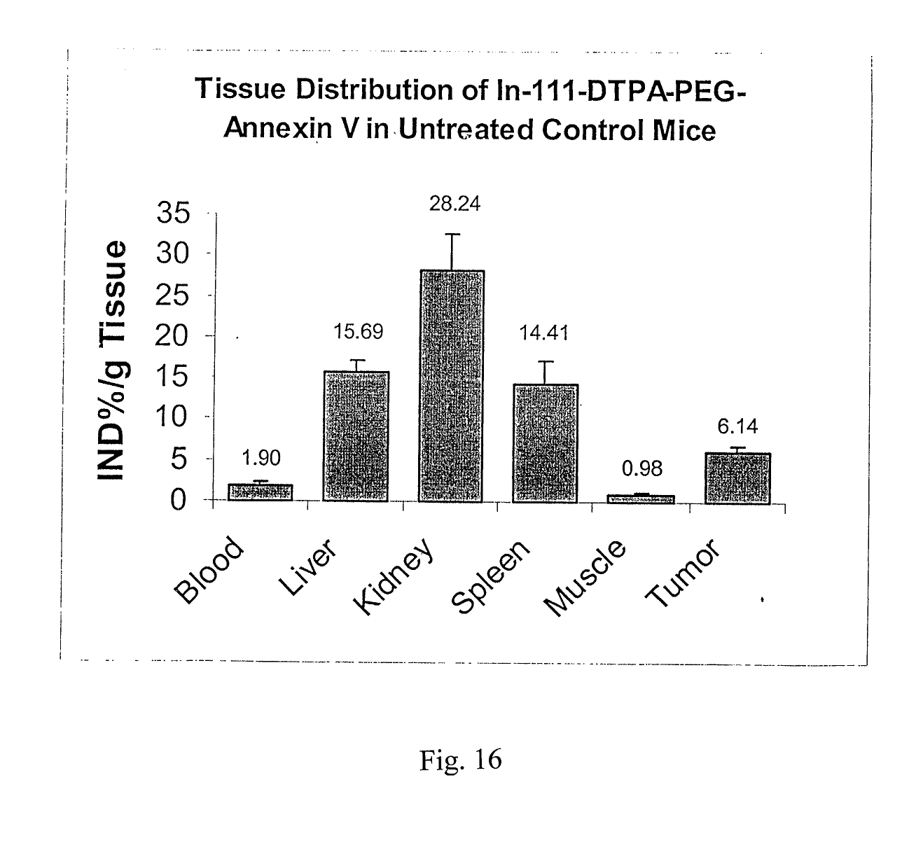

Results were as follows. The tissue distribution of 1:30 prep of ¹¹¹In-DTPA-PEG-AV at 48 hours after the injection of radiotracer in untreated control mice and in mice treated with PG-TXL (100 mg eq./kg) or C225 (50 mg/kg) 4 days before the injection of the radiotracer are presented in FIGS. 16-18.

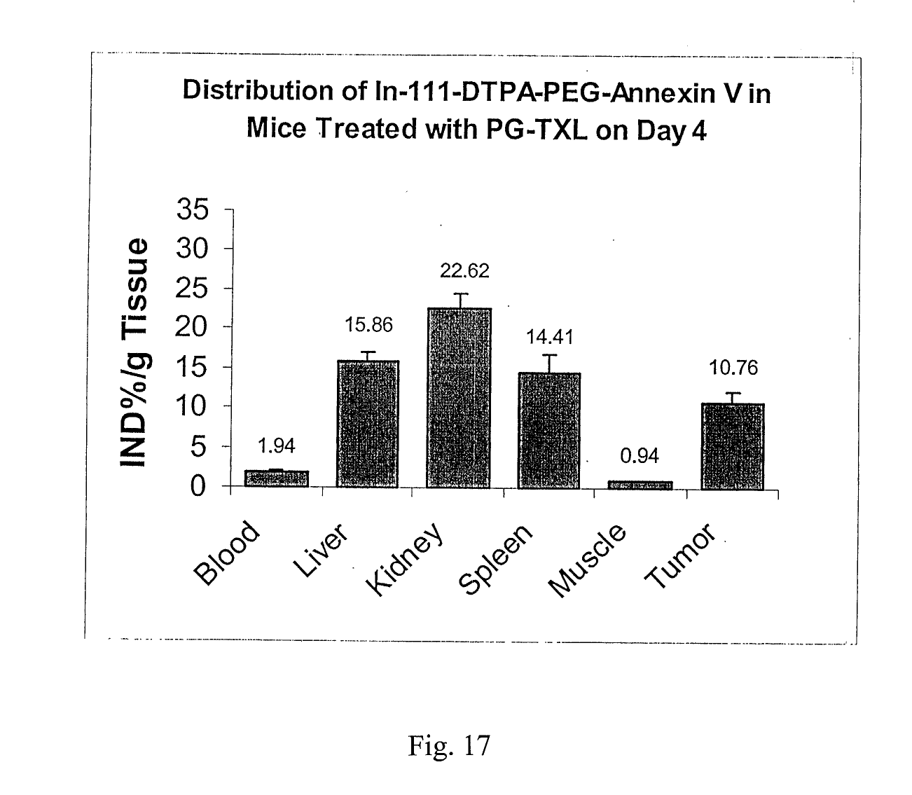

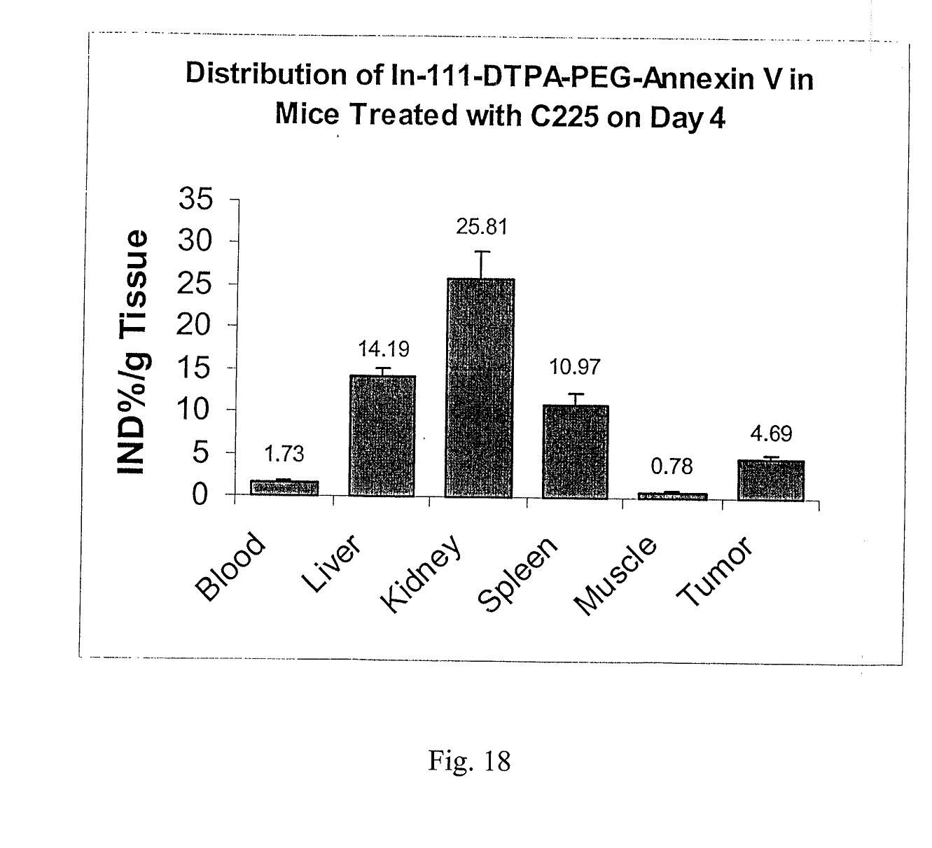

In FIGS. 16-18, the y-axis represents IND %/g tissue. The bars along the x-axis from left to right represent blood, liver, kidney, spleen, muscle and tumor, respectively. FIG. 16 shows tissue distribution of ¹¹¹In-DTPA-PEG-AV in untreated control mice. FIG. 17 shows distribution of ¹¹¹In-DTPA-PEG-AV in mice treated with PG-TXL on day 4. FIG. 18 shows distribution of ¹¹¹In-DTPA-PEG-AV in mice treated with C225 on day 4.

Treatment with PG-TXL resulted in a highly significant increase in the uptake of ¹¹¹In-DTPA-PEG-AV in the tumor 4 days after drug injection (p=0.0009). The percentage of injected dose (% IND) per gram of tumor increased from 6.14 to 10.76, a 75% increase. Treatment with C225, on the other hand, resulted in a significant decease in the uptake of the radiotracer in the tumor (p=0.028). PG-TXL induced apoptosis in MDA-MB468 tumors was time dependent. At 1 day after drug treatment, a slight but non-significant increase of the radiotracer in the tumor was observed (p=0.26). Combination therapy with PG-TXL and C225 also resulted in a significant increase in the uptake of ¹¹¹In-DTPA-PEG-AV at 4 days after treatment (p=0.029, data not shown).

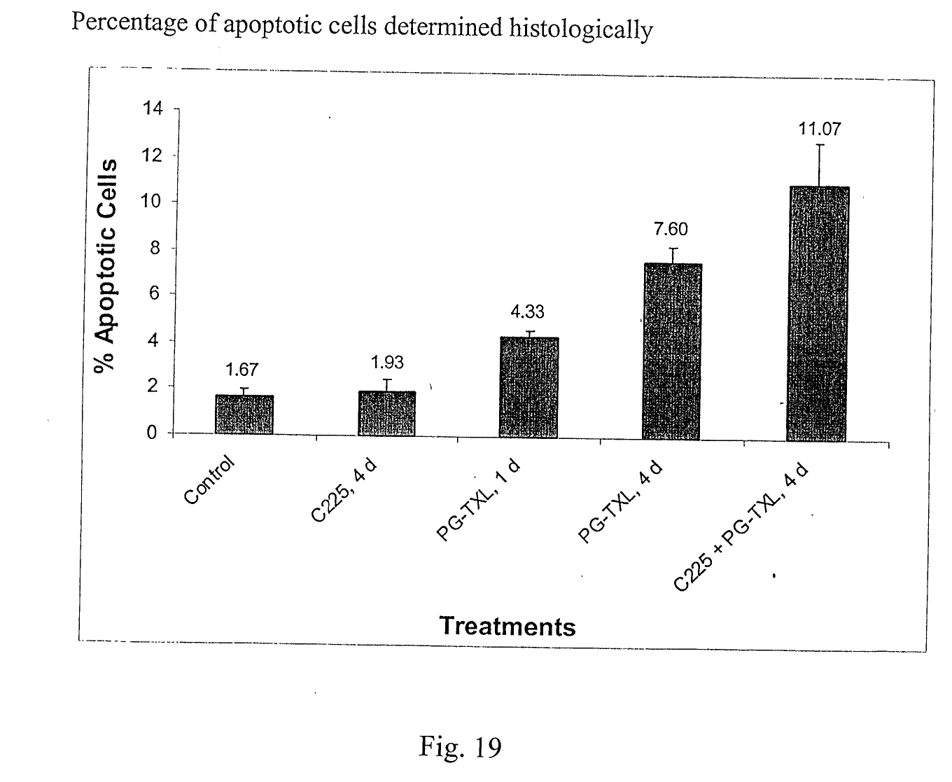

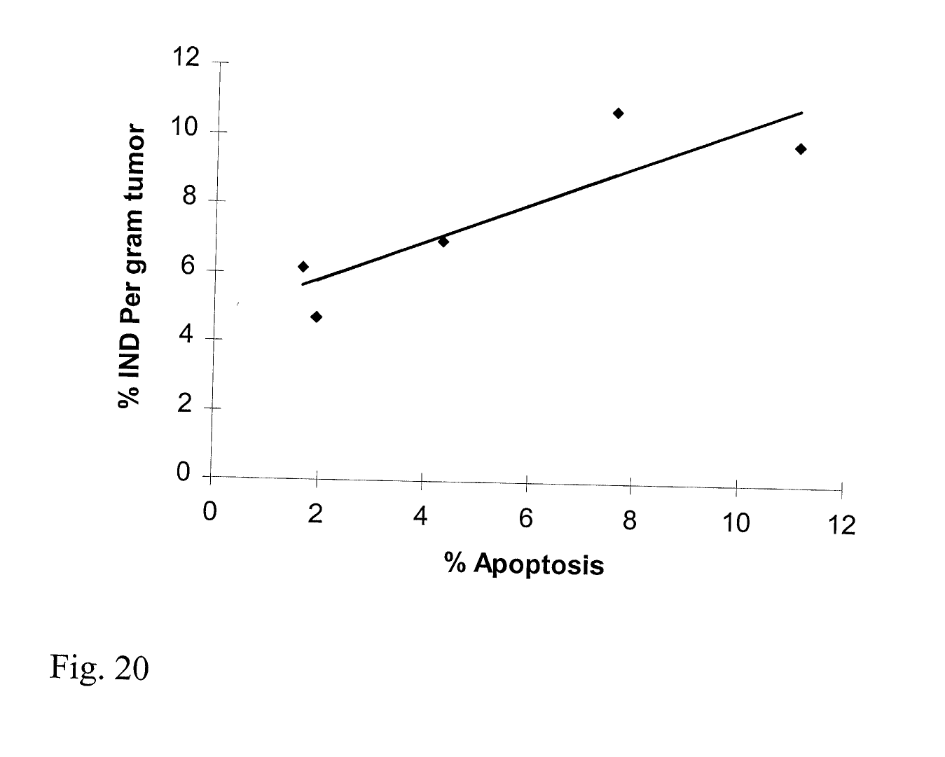

To correlate the findings obtained with ¹¹¹In-DTPA-PEG-AV to the apoptotic index obtained from a conventionally accepted standard, the percentage of apoptotic cells was quantified histologically in tumor samples removed at different times after treatments. The data are summarized in FIG. 19, a bar graph showing the percentage of apoptotic cells determined histologically. In FIG. 19 the y-axis represents the % apoptotic cells. The bars along the x-axis, from left to right, represent the control mice, and mice treated with: C225, 4d; PG-TXL, Id; PG-TXL 4d; and C225+PG-TXL 4d, respectively. The correlation coefficiency (r²) was analyzed by a simple regression model. The resulting data indicate that the uptake of the radiotracer in the tumor was highly correlated to apoptotic index determined histologically with r² value of 0.77 (FIG. 20). In FIG. 20, the y-axis represents the % IND per gram tumor; the x-axis represents the % apoptosis.

Gamma images of mice acquired at 48 hours after injection of the radiotracer in mice treated with PG-TXL 4 days prior to the injection of the radiotracer were obtained. The gamma imaging study allowed visualization of changes associated with drug treatment. The enhancement of the contrast in the tumor could be clearly visualized.

### Example 27

### Autoradiography and TUNEL Assay

Two tumors removed from each group of mice at the end of the gamma imaging X5 session were rapidly frozen, processed, and 8-μm sections were made. The sliced sections were fixed in cold acetone, dried, and exposed to a multipurpose storage phosphor screen for 10 days. A Cyclone Storage Phosphor System (Packard Instrument Company, Inc., Meriden, Conn.) was used to obtain autoradiographic images. The radioactivity at selected field of view is expressed as digital light unit per millimeter square (DLU/mm²).

DNA fragmentation was analyzed by terminal deoxynucleotidyl transferase (TdT)-uridine nick end labeling (TUNEL) using a commercial kit according to the manufacturer provided protocol (Promega, Madison, Wis.). Frozen tissue sections (8 μm) were fixed with 4% paraformaldehyde (methanol-free) for 10 minutes at room temperature. The sections were washed with PBS two times (5 minutes each) and incubated with equilibration buffer (Promega) for 10 minutes at room temperature. The equilibration buffer was removed and reaction buffer containing equilibration buffer, nucleotide mix, and TdT enzyme was added to the tissue. Slides were incubated for 1 hour at 37° C. in the dark. The TUNEL reaction was terminated by immersing the slides in 2× SSC (17.5 g of NaCl and 8.8 g of sodium citrate, 1 liter H₂O, pH 7.0) for 15 minutes. The slides were washed three times (5 minutes each) to remove unincorporated fluorescein-dUTP. To prevent photobleaching, Vectashield mounting medium (Vector Laboratories, Inc., Burlingame, Calif.) was used to mount cover slips. Images were recorded using an Olympus fluorescence microscope (Olympus, Melville, N.Y.) with 520 nm filter and 20× object lens. Apoptotic cells per slide view were quantified by AAB colony counting software (Advanced American Biotechnology, Fullerton, Calif.) and expressed as number per field of view (0.016 mm²).

The intratumoral distribution of ¹¹¹In-DTPA-PEG-AV in tumors of untreated control mice and mice 4 days after treatments with PG-TXL, C225, or both drugs combined were compared. Radioactivity was localized mainly in the periphery of the tumor in control mice. There was little activity in the central zone of the tumor. When the mice were treated with PG-TXL, the activity was concentrated more in the central area of the tumor. On the other hand, treatment with C225 alone caused reduced distribution of ¹¹¹In-DTPA-PEG-AV in the peripheral zone of the tumor as compared to controls. Combined PG-TXL and C225 therapy caused similar intratumoral distribution pattern of ¹¹¹In-DTPA-PEG-AV as those treated with PG-TXL alone. The data confirm that treatment with PG-TXL alone or combined PG-TXL and C225 caused increased apoptosis, whereas treatment with C225 alone caused reduced apoptotic response. The findings also underscore that tumors are heterogeneous, as are their responses to treatments.

To further confirm that the distribution of radioactivity or ¹¹¹In-DTPA-PEG-AV within MB-468 tumors corresponds to distribution of apoptotic cells, the same slides used for autoradiographic studies were stained with TUNEL to visualize the intratumoral distribution of apoptotic cells. The “hot spots” in autoradiographs of tumors co-localized with strong, positive TUNEL staining, and the “cold spots” co-localized with weak TUNEL staining. Even the boundary between “hot” and “cold” area in the autoradiographs corresponded to distinguishable areas of strong and weak stains in the TUNEL stained slide.

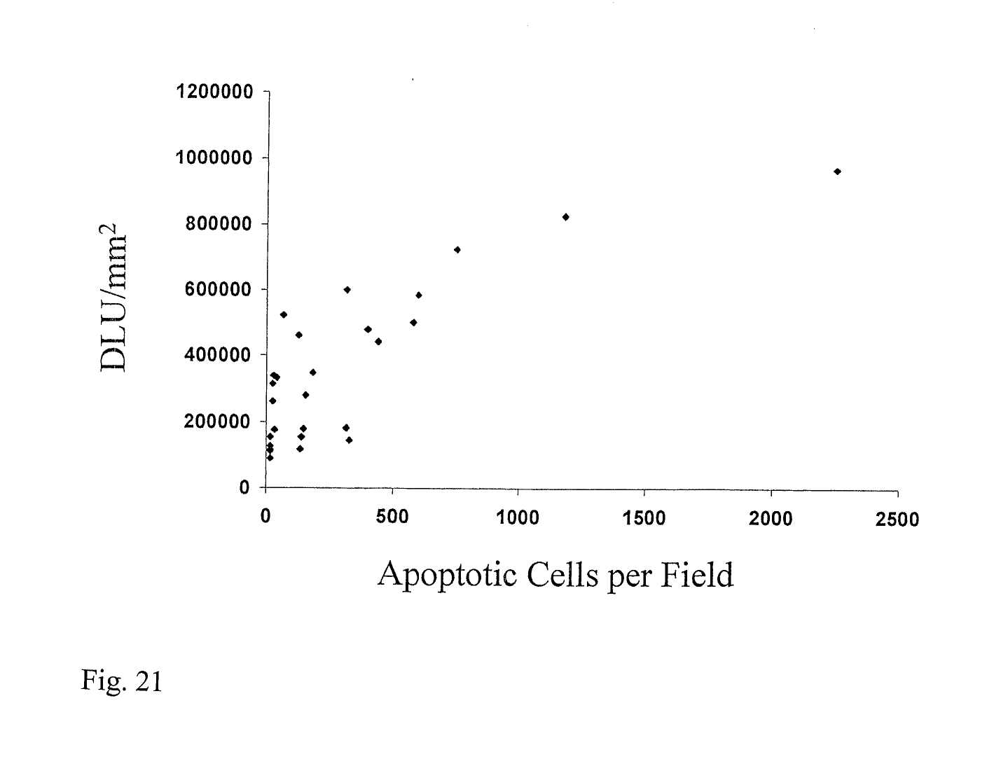

Quantitative analysis demonstrated significant correlation (r=0.71, p=0.0001) between radioactivity in autoradiographs and the corresponding fluorescent intensity in the TUNEL stained slide. Results are shown in FIG. 21. Specifically, FIG. 21 is a graph showing the correlation between radioactivity measured from phosphors screen images (DLU/mm²; y-axis) and apoptotic index determined from TUNEL assay (# Apoptotic Cells/Field; x-axis). These data further support that ¹¹¹In-DTPA-PEG-AV can be used to measure tumor apoptosis.

The foregoing data demonstrate, among other things, that ¹¹¹n-DTPA-PEG-AV and similar conjugates may be used to visualize apoptosis in cells and are potentially a useful tool in non-invasive assessment of early responses after chemo- and radiotherapy. Most chemotherapeutic drugs exert their antitumor activity by inducing apoptosis, and recent data suggest that early cell death may be an important predictor of the success of chemotherapy. As such, being able to noninvasively assess apoptosis using the novel conjugates of the invention will provide highly useful information to the physician, allowing evaluation of early treatment responses and better planning and monitoring of treatment of a patient.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. Not all embodiments of the invention will include all the specified advantages. The specification and examples should be considered exemplary only with the true scope and spirit of the invention indicated by the following claims.

We claim:

1. A conjugate molecule comprising:
a ligand bonded to a polymer;
a chelating agent bonded to the polymer;
and a radioisotope chelated to the chelating agent.

2. The molecule of claim 1, wherein the ligand is covalently bonded to the polymer and the chelating agent is covalently bonded to the polymer.

3. The molecule of claim 1, wherein the ligand is a peptide, a protein, an antibody or an antibody fragment.

4. The molecule of claim 1, wherein the ligand is selected from the group consisting of C225, Herceptin, Rituxan, a phage library antibody, anti-CD, DC101, an antibody to integrin alpha v-beta 3, LM609, an antibody to VEGF, an antibody to VEGF receptor, F(ab′)₂, Fab′, ScFv fragment, c7E3Fab, a growth factor, VEGF-A, VEGF-B, VEGF-C, VEGF-D, PDGF, Angiopoietin-1, Angiopoietin-2, HGF, EGF, bFGF, cyclic CTTHWGFTLC, cyclic CNGRC, cyclic RGD-4C, annexin V, an interferon, a tumor necrosis factor, endostatin, angiostatin and thrombospondin.

5. The molecule of claim 1, wherein the ligand is annexin V.

6. The molecule of claim 1, wherein the ligand is C225.

7. The molecule of claim 1, wherein the ligand is an antibody.

8. The molecule of claim 1, wherein the ligand is a monoclonal antibody.

9. The molecule of claim 1, wherein the ligand is a polyclonal antibody.

10. The molecule of claim 1, wherein the polymer is polyethylene glycol.

11. The molecule of claim 10, wherein the polyethylene glycol has a number average molecular weight of about 1,000 daltons to about 100,000 daltons.

12. The molecule of claim 1, wherein the polymer is a polysaccharide.

13. The molecule of claim 12, wherein the polysaccharide has a number average molecular weight of about 1,000 daltons to about 150,000 daltons.

14. The molecule of claim 1, wherein the polymer is a polyamino acid.

15. The molecule of claim 14, wherein the polyamino acid has a number average molecular weight of about 1,000 daltons to about 150,000 daltons.

16. The molecule of claim 1, wherein the polymer is poly(1-glutamic acid), poly(d-glutamic acid), poly(d1-glutamic acid), poly(1-aspartic acid), poly(d-aspartic acid), poly(d1-aspartic acid), polylysine, a polysaccharide, dextran, polypropylene oxide (PPO), polyvinyl pyrolidone, polyvinyl alcohol, polyethylene glycol, hyaluronic acid, chitosan, dextran, polyacrylic acid, poly(2-hydroxyethyl 1-glutamine) or a carboxymethyl dextran.

17. The molecule of claim 1 wherein the polymer is a copolymer between two or more of the following polymers: poly(1-glutamic acid), poly(d-glutamic acid), poly(d1-glutamic acid), poly(1-aspartic acid), poly(d-aspartic acid), poly(d1-aspartic acid), polylysine, a polysaccharide, dextran, polypropylene oxide (PPO), polyvinyl pyrolidone, polyvinyl alcohol, polyethylene glycol, hyaluronic acid, chitosan, dextran, polyacrylic acid, poly(2-hydroxyethyl 1-glutamine) and a carboxymethyl dextran.

18. The molecule of claim 1, wherein the chelating agent is selected from the group consisting of DTPA, EC, DMSA, EDTA, Cy-EDTA, EDTMP, DTPA, CyDTPA, Cy2DTPA, BOPTA, DTPA-MA, DTPA-BA, DTPMP, DOTA, TRITA, TETA, DOTMA, DOTA-MA, HP-DO3A, pNB-DOTA, DOTP, DOTMP, DOTEP, DOTPP, DOTBzP, DOTPME, HEDP, DTTP, an N₃S triamidethiol, DADS, MAMA, DADT, an N₂S₄ diaminetetrathiol, an N₂P₂ dithiol-bisphosphine, a 6-hydrazinonicotinic acid, a propylene amine oxime, a tetraamine and a cyclam.

19. The molecule of claim 1, wherein the chelating agent is DOTA.

20. The molecule of claim 1, wherein the chelating agent is DTPA.

21. The molecule of claim 1, wherein the radioisotope is selected from the group consisting of ¹¹¹In, ⁶⁷Ga, ⁶⁸Ga, ⁸²Rb ⁸⁶Y, ⁹⁰Y, ^{99m}Tc, ⁶⁴Cu, ⁶⁷Cu, ¹⁹³Pt, ^{113m}In and ²⁰¹Tl.

22. The molecule of claim 1, wherein the radioisotope is ¹¹¹In.

23. A composition comprising the conjugate molecule of claim 1 and a pharmaceutically acceptable carrier.

24. A method for selectively delivering a diagnostic agent to apoptotic cells in a patient comprising the step of administering a conjugate molecule to the patient having apoptotic cells, wherein the conjugate molecule comprises a ligand bonded to a polymer, wherein said ligand is annexin V, a chelating agent bonded to the polymer, and a radioisotope chelated to the chelating agent.

25. The method of claim 24, wherein the ligand is covalently bonded to the polymer and the chelating agent is covalently bonded to the polymer.

26. The method of claim 24, wherein the administering step comprises intravascular, intraperitoneal, intramuscular or intratumoral injection.

27. The method of claim 24, wherein the patient is a mammal.

28. The method of claim 24, wherein the patient is a human.

29. The method of claim 24, wherein the apoptotic cells are present following treatment of a target tissue.

30. The method of claim 24, wherein the target tissue is a tumor.

31. A method of treating a patient suspected of having a tumor, the method comprising administering a therapeutically effective amount of a conjugate molecule to the patient, wherein the conjugate molecule comprises a ligand bonded to a polymer, a chelating agent bonded to the polymer, and a radioisotope chelated to the chelating agent, and wherein said ligand has affinity for the tumor.

32. The method of claim 31, wherein the ligand is covalently bonded to the polymer and the chelating agent is covalently bonded to the polymer.

33. The method of claim 31, wherein the radioisotope is ⁹⁰Y, ⁶⁴Cu or ⁶⁷Cu.

34. The method of claim 31, wherein the patient is a mammal.

35. The method of claim 31, wherein the patient is a human.

36. The method of claim 31, wherein the ligand is an antibody or a protein.

37. The method of claim 31 wherein the ligand is Herceptin or C225.

38. The method of claim 31 wherein the ligand is annexin V.

39. The method of claim 31, wherein the polymer is polyethylene glycol.

40. The method of claim 31, wherein the chelating agent is DTPA.

41. The method of claim 31, wherein the chelating agent is DOTA.

42. The method of claim 31, wherein the administering step comprises intravascular, intraperitoneal, intramuscular or intratumoral injection.

43. The method of claim 31, wherein the tumor is a solid tumor.

44. The method of claim 31, wherein the tumor is a breast cancer tumor, an ovarian cancer tumor, a colon cancer tumor, a lung cancer tumor, a head and neck cancer tumor, a brain tumor, a liver cancer tumor, a pancreatic tumor, a bone cancer tumor or a prostate cancer tumor.

45. A method of visualizing tumors, the method comprising:
administering a conjugate molecule to a patient suspected of having a tumor; and
detecting the conjugate molecule; wherein the conjugate molecule comprises a ligand bonded to a polymer, a chelating agent bonded to the polymer, and a radioisotope chelated to the chelating agent, and wherein said ligand has affinity for the tumor.

46. The method of claim 45, wherein the ligand is covalently bonded to the polymer and the chelating agent is covalently bonded to the polymer.

47. The method of claim 45, wherein the ligand is an antibody or protein.

48. The method of claim 45 wherein the ligand is Herceptin or C225.

49. The method of claim 45 wherein the ligand is annexin V.

50. The method of claim 45, wherein the polymer is polyethylene glycol.

51. The method of claim 45, wherein the chelating agent is DTPA.

52. The method of claim 45, wherein the chelating agent is DOTA.

53. The method of claim 45, wherein the radioisotope is ¹¹¹In.

54. The method of claim 45, wherein the radioisotope is ⁶⁴Cu.

55. The method of claim 45, wherein the administering step comprises intravascular, intraperitoneal, intramuscular or intratumoral injection.

56. The method of claim 45, wherein the detecting step comprises detection of the radioisotope by radioscintigraphy, single photon emission computed tomography or positron emission tomography.

57. The method of claim 45, wherein the patient is a mammal.

58. The method of claim 45, wherein the patient is a human.

59. The method of claim 45, wherein the tumor is a solid tumor.

60. The method of claim 45, wherein the tumor is a breast cancer tumor, an ovarian cancer tumor, a colon cancer tumor, a lung cancer tumor, a head and neck cancer tumor, a brain tumor, a liver cancer tumor, a pancreatic tumor, a bone cancer tumor or a prostate cancer tumor.

61. A method for visualizing apoptotic cells in a patient comprising the steps of:
administering a conjugate molecule to the patient having apoptotic cells, wherein the conjugate molecule comprises a ligand bonded to a polymer, wherein said ligand is annexin V, a chelating agent bonded to the polymer, and a radioisotope chelated to the chelating agent; and
detecting the conjugate molecule.

62. The method of claim 61 wherein the apoptotic cells are associated with a disease or condition selected from the group consisting of an acute organ transplant rejection, an inflammatory disease, an infectious disease, a regenerative tissue, a post-surgery tissue, a post-trauma tissue, a hypoxic-ischaemic cerebral reperfusion injury, a toxic effect of a chemotherapeutic agent to normal tissue, sickle cell disease, thalassemia, multiple sclerosis and rheumatoid arthritis.

63. The method of claim 61, wherein the ligand is covalently bonded to the polymer and the chelating agent is covalently bonded to the polymer.

64. The method of claim 61, wherein the detecting step comprises detection of the radioisotope by radioscintigraphy, single photon emission computed tomography or positron emission tomography.

65. The method of claim 61, wherein the administering step comprises intravascular, intraperitoneal, intramuscular or intratumoral injection.

66. The method of claim 61, wherein the patient is a mammal.

67. The method of claim 61, wherein the patient is a human.

68. The method of claim 61, wherein the apoptotic cells are present in a target tissue.

69. The method of claim 61, wherein the target tissue is a tumor.

70. A method of visualizing tumors or apoptotic cells, the method comprising:
administering a conjugate molecule to a patient suspected of having a tumor or apoptotic cells; and
detecting the conjugate molecule, wherein the conjugate molecule comprises a ligand bonded to a polymer and a near-infrared dye bonded to the polymer, and wherein said ligand has affinity for the tumor or apoptotic cells.

71. The method of claim 70 wherein the near-infrared dye is ICG or an ICG derivative.

72. The method of claim 70 wherein the detecting step comprises detection of the near-infrared dye by a near-infrared camera.

73. A method for synthesizing a ligand-polymer-chelating agent-diagnostic agent conjugate molecule comprising:
providing an SCN-polymer-chelating agent precursor, wherein said polymer is covalently bonded to the chelating agent and said SCN group is covalently bonded to the polymer;
combining a ligand with said SCN-polymer-chelating agent precursor to form a ligand-polymer-chelating agent conjugate; and
combining said ligand-polymer-chelating agent conjugate with a diagnostic agent to form said ligand-polymer-chelating agent-diagnostic agent conjugate molecule, wherein the ligand is covalently bonded to the polymer, the polymer is covalently bonded to the chelating agent, and the diagnostic agent is chelated to the chelating agent.

74. A method for synthesizing a conjugate molecule comprising:
providing a polymer conjugate-SCN precursor, wherein said SCN group is covalently bonded to the polymer conjugate; and
combining a ligand with said polymer conjugate-SCN precursor to form a ligand-polymer conjugate molecule, wherein said ligand is covalently bonded to said polymer.

75. The method of claim 74, wherein the ligand comprises a primary amino group.

76. The method of claim 74, wherein said polymer conjugate comprises a polymer covalently bonded to a chelating agent.

77. The method of claim 76, further comprising combining said ligand-polymer conjugate molecule with a diagnostic agent to form a ligand-polymer-chelating agent-diagnostic agent conjugate molecule.

78. The method of claim 77, wherein the diagnostic agent is a radioisotope.

79. The method of claim 78, wherein the radioisotope is selected from the group consisting of ¹¹¹In, ⁶⁷Ga, ⁶⁸Ga, ⁸²Rb, ⁸⁶Y, ⁹⁰Y, ^{99m}TC, ⁶⁴Cu, ⁶⁷Cu, ¹⁹³Pt, ^{113m}In and ²⁰¹Tl.

80. The method of claim 79, wherein the radioisotope is ¹¹¹In.

81. The method of claim 79, wherein the radioisotope is ⁶⁴Cu.

82. The method of claim 74, wherein the polymer conjugate comprises a polymer covalently bonded to a therapeutic agent.

83. The method of claim 74 wherein the therapeutic agent is a diagnostic agent.

84. The method of claim 83 wherein the diagnostic agent is a dye molecule.

85. The method of claim 74, wherein, the ligand is a peptide, a protein, an antibody or an antibody fragment.

86. The method of claim 74, wherein the ligand is selected from the group consisting of C225, Herceptin, Rituxan, a phage library antibody, anti-CD, DC10l, an antibody to integrin alpha v-beta 3, LM609, an antibody to VEGF, an antibody to VEGF receptor, F(ab′)₂, Fab′, ScFv fragment, c7E3Fab, a growth factor, VEGF-A, VEGF-B, VEGF-C, VEGF-D, PDGF, Angiopoietin-1, Angiopoietin-2, HGF, EGF, bFGF, cyclic CTTHWGFTLC, cyclic CNGRC, cyclic RGD-4C, annexin V, an interferon, a tumor necrosis factor, endostatin, angiostatin and thrombospondin.

87. The method of claim 74, wherein the ligand is annexin V.

88. The method of claim 76, wherein the chelating agent is selected from the group consisting of DTPA, EC, DMSA, EDTA, Cy-EDTA, EDTMP, DTPA, CyDTPA, Cy2DTPA, BOPTA, DTPA-MA, DTPA-BA, DTPMP, DOTA, TRITA, TETA, DOTMA, DOTA-MA, HP-DO3A, pNB-DOTA, DOTP, DOTMP, DOTEP, DOTPP, DOTBzP, DOTPME, HEDP, DTTP, an N₃S triamidethiol, DADS, MAMA, DADT, an N₂S₄ diaminetetrathiol, an N₂P₂ dithiol-bisphosphine, a 6-hydrazinonicotinic acid, a propylene amine oxime, a tetraamine and a cyclam.

89. The method of claim 76, wherein the chelating agent is DTPA.

90. The method of claim 76, wherein the chelating agent is DOTA.

91. The method of claim 74, wherein the polymer conjugate-SCN precursor comprises a polymer selected from the group consisting of poly(1-glutamic acid), poly(d-glutamic acid), poly(d1-glutamic acid), poly(1-aspartic acid), poly(d-aspartic acid), poly(d1-aspartic acid), polylysine, a polysaccharide, dextran, polypropylene oxide (PPO), polyvinyl pyrolidone, polyvinyl alcohol, polyethylene glycol, hyaluronic acid, chitosan, dextran, polyacrylic acid, poly(2-hydroxyethyl 1-glutamine) and a carboxymethyl dextran.

92. The method of claim 74 wherein the polymer conjugate-SCN precursor comprises a copolymer between two or more of the following polymers: poly(1-glutamic acid), poly(d-glutamic acid), poly(d1-glutamic acid), poly(1-aspartic acid), poly(d-aspartic acid), poly(d1-aspartic acid), polylysine, a polysaccharide, dextran, polypropylene oxide (PPO), polyvinyl pyrolidone, polyvinyl alcohol, polyethylene glycol, hyaluronic acid, chitosan, dextran, polyacrylic acid, poly(2-hydroxyethyl 1-glutamine) and a carboxymethyl dextran.

93. The method of claim 74, wherein the polymer conjugate-SCN precursor comprises a polymer selected from the group consisting of polyethylene glycol, poly (1-glutamic acid), dextran, polyvinyl alcohol, polyethylene oxide-polypropylene oxide copolymer and copolymers between two or more thereof.

94. The method of claim 74, wherein the polymer is polyethylene glycol.

95. A method synthesizing a conjugate molecule comprising:
providing a polymer conjugate, wherein the polymer conjugate comprises at least one thio (SH) group covalently conjugated to the polymer conjugate;
providing a ligand, the ligand comprising at least one thio reactive group; and
combining the polymer conjugate and the ligand to form a ligand-polymer conjugate molecule.

96. The method of claim 95 wherein the ligand is pretreated with an agent to introduce the at least one thio-reactive group.

97. The method of claim 96 wherein the agent is vinyl sulfone or maleimide.

98. The method of claim 95 wherein the step of providing the polymer conjugate comprises the steps of:
obtaining a precursor polymer conjugate having a protected thio group; and
treating the precursor polymer with a deblocking agent to release a free thio group

99. The method of claim 95 wherein the polymer conjugate comprises a polymer covalently bonded to a chelating agent.

100. The method of claim 99 further comprising combining the ligand-polymer conjugate molecule with a diagnostic agent to form a conjugate molecule construct comprising a ligand-polymer-chelating agent-diagnostic agent construct.

101. A method for synthesizing a conjugate molecule comprising the steps of:
providing a polymer conjugate and a ligand, wherein one of the polymer conjugate or the ligand comprises a thio group, and the other of the polymer conjugate or the ligand comprises a thio reactive group; and
combining the polymer conjugate and the ligand to form a ligand-polymer conjugate molecule, wherein the ligand is covalently bonded to the polymer by a thioether (S—C) bond.

102. The method of claim 101 wherein the thio group is attached to the ligand and the thio reactive group is attached to the polymer conjugate, and the polymer conjugate is prepared by attaching SPDP or maleimide to the polymer conjugate.

103. The method of claim 101 wherein the thio group is attached to the polymer conjugate and the thio reactive group is attached to the ligand and the ligand is pretreated with maleimide or vinyl sulfone to introduce the thio reactive group.

104. The method of claim 101 wherein the polymer conjugate comprises a polymer bonded to a diagnostic agent.

105. The method of claim 101 wherein the polymer conjugate comprises a polymer bonded to a chelating agent.
